SEQUENCE LISTING (28 sequences)

SEQ ID NO: 1 - protein, 209 aa, Porphyromonas gingivalis
QSAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFVYNAAAYARKGFVGQELTQVEMLGTMRGVRIAALTINPVQYDVVANQLKVRNNIEIEVSFQGADEVATQRLYDASFSPYFETAYKQLFNR

SEQ ID NO: 2 - protein, 205 aa, Porphyromonas gingivalis
QPAERGRNPQVRLLSAEQSMSKVQFRMDNLQFTDVQTSKGVAQVPTFTEGVNISEKGTPILPILSRSLAVSETRAMKVEVVSSKFIEKKDVLIAPSKGVISRAENPDQIPYVYGQSYNEDKFFPGEIATLSDPFILRDVRGQVVNFAPLQYNPVTKTLRIYTEIVVAVSETAEAGQNTISLVKNSTFTGFEDIYKSVFMNYEATR

SEQ ID NO: 3 - protein, 203 aa, Porphyromonas gingivalis
QTELGRNPNVRLLESTQQSVTKVQFRMDNLKFTEVQTPKGMAQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGR

SEQ ID NO: 4 - protein, 209 aa, Porphyromonas gingivalis
QSAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFAYNAAAYARKGFVGQELTQVEMLGTMRGVRIAALTINPVQYDVVANQLKVRNNIEIEVSFQGADEVATQRLYDASFSPYFETAYKQLFNR

SEQ ID NO: 5 - protein, 209 aa, Porphyromonas gingivalis
QNAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFVYNAAAYARKGFVGQELTQVEMLGTMRGVRIAALTINPVQYDVVANQLKVRNNIEIEVSFQGADEVATQRLYDASFSPYFETAYKQLFNR

SEQ ID NO: 6 - protein, 205 aa, Porphyromonas gingivalis
QPAERGRNPQVRLLSAEQSMSKVQFRMDNLQFTGVQTSKGVAQVPTFTEGVNISEKGTPILPILSRSLAVSETRAMKVEVVSSKFIEKKDVLIAPSKGVISRAENPDQIPYVYGQSYNEDKFFPGEIATLSDPFILRDVRGQVVNFAPLQYNPVTKTLRIYTEIVVAVSETAEAGQNTISLVKNSTFTGFEDIYKSVFMNYEATR

SEQ ID NO: 7 - protein, 205 aa, Porphyromonas gingivalis
QPAERGRNPQVRLLSAEQSMSKVQFRMDNLQFTGVQTSKGVAQVPTFTEGVNISEKGTPILPILSRSLAVSETRAMKVEVVSSKFIEKKDVLIAPSKGVISRAENPDQIPYVYGQSYNEDKFFPGENATLSDPFILRDVRGQVVNFAPLQYNPVTKTLRIYTEIVVAVSETAEAGQNTISLVKNSTFTGFEDIYKSVFMNYEATR

SEQ ID NO: 8 - protein, 203 aa, Porphyromonas gingivalis
QTELGRNPNVRLLESTQQSVTKVQFRMDNLKFTEVQTPKGIAQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGR

SEQ ID NO: 9 - protein, 203 aa, Porphyromonas gingivalis
QTELGRNPNVRLLESTQQSVTKVQFRMDNLKFTEVQTPKGMGQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGR

SEQ ID NO: 10 - protein, 215 aa, Porphyromonas gingivalis
QSQTWHGDPDSVAALPSIGIQESSCTRITFEVVFPGFYSVEKREGNQVFQRISMPGCGSFGNLGEAELPVLKKMIAVPEFSTANVAVKIKETETFDNYNIYPNPTYVVEELPEGGTYLVEAFAINNDYYSQNVSLPSTHYVYSQDGYFRSQRFIEVTLYPFRYNPVRQEILFAKKIEVTITFDNPQPPLQKNTGIFNKVASSAFINYEADGKSAI

SEQ ID NO: 11 - protein, 191 aa, Candidatus cloacamonas acidaminovorans
VQLLEKSDTGLSIAYAVDELQFREINTKEGIFTELNALNYTTTNVTGLPALPLMRQLISVPLGANVTANIVSSSAKVINLDEQGVLYPLMPRQESVSKSADLEQLPFEVNRDFYNANMWTDNPSITVTEIGMMRGTRIFAVDFVPIKYNPVLKKIEVIYQAEVKVAFSGANFIATNELQAKTYSPAFEGVF

SEQ ID NO: 12 - protein, 182 aa, Candidatus cloacamonas acidaminovorans
ELQFRNQEYFLEEVETPSGKFTRINMDGFGFSQRIGEPQLPVYSKLIAVPVGAKVEFSFGRNTEITLQKSETLITNRIYPAQPSVSKSQDPALISFELKTDIYSKNEFYSGELFSVAEIGFLRGVRIFRIDYEPMRYNPVSGELKINTELNVQVKFINADFSATQDLLARTASYEFDSLYGK

SEQ ID NO: 13 - protein, 60 aa, Candidatus cloacamonas acidaminovorans
ENRLYPSQQWQFLGTQYFRGYQIALFNVYPYRYNPVTQKLYVSSQISISINSEFSEEEAC

SEQ ID NO: 14 - protein, 143 aa, Candidatus cloacamonas acidaminovorans
EAGMPQVPVLARSLIIPATAKMHLNITNSEYVELTLPVAPSKGNLTRDIDPATIPYTFADFYQSGESYPAEIAYLTEPFILRDYRGITVRFQPFIYYPATQTLRVYTKLNISVYTQGTDLTNALLSPKTSYSRYFESTYQNMF

SEQ ID NO: 15 - protein, 153 aa, Candidatus cloacamonas acidaminovorans
EWYQIRLPKEGITQDKGYPELPVFNRSIIIPDQALMAIEVFDLEFKDYPIKVAPSKGVITRDINPATIPYTFGNVYQENSFYPQKMVALSEPYILRDFRGITVLTTPFAYNPVTGTLRVYTSYKVKVYNQGNDTVNSFNRSRTSVSRSFCPLY

SEQ ID NO: 16 - protein, 163 aa, Candidatus cloacamonas acidaminovorans
VSESQDILAINFELPEYELTKVTINGQNWERIVCSDGSYFSNEGFPQLIMFSTAIAVPVDGDYSFSIKSSEPKTLTNINIIPSSTLLIEGEGVNYNNKPDYKAYANRELYPVSLAQKGEPAFIGNRKFIPLLIYPFQYRAQSKELIVHSKISITVYISGTKNA

SEQ ID NO: 17 - protein, 129 aa, Candidate division WS3
LEVEGADAMGVPGAPDLPVVRLTLAVPECRDIQLAVSTGGRSSERGVNVIPALTTVETEEGEVSRYEYVEGDHYARGGLWPSSVATMTDPRWLSRQRVVHVEIYPCQVDPVEGTLVSHDTIEVRLSFTG

SEQ ID NO: 18 - protein, 176 aa, Candidatus cloacamonas acidaminovorans
SQNAGKIKLQLQVPELHIEDIDNSNFKILSMQGAETTAETGFPELPVFSAWIAIPPKGDIEIKVTGGEIITQKGFIPKPVFATKEQEIASEYNKIAYRSASLYPANSYSYSQPQIIRDFRVVQITLNPVHYVAETQELKIQKQMEVEIEIKDRPGINEMDEYNGYSYAFTNLYESM

SEQ ID NO: 19 - protein, 174 aa, Candidatus cloacamonas acidaminovorans
IKEINDSEIRIQFTLPQWEIEQINVKNEIRKKVKVQETPYLFIDEEETLPVFSTMIAIPNRGGVDLLVSNSSKSSINEFTANFDAALNRESQQGRFTDIHYPSANVVISEPQILRDFRVVNLNIYPFQYDRINRKLLVSENLDIKLTFNSRPSINELNSSSYISCSFDSIYRGL

SEQ ID NO: 20 - protein, 184 aa, Candidatus cloacamonas acidaminovorans
LGAKNSSSLQVNFTLPEYSVQEETYGGAVYHKIMLPLSGTLMETGMPELPVVCTSIAIPHTGGVNIEVLSTQQTVIPNFLPYPVQQGNSSESPKGFIINNEYYNSGNNYPEMLIEYSEPSILRDFRIITIQINPFFYNPQTGELTVKHNIDFCLNFTQEQGINELPNEPANISASFDKIYDSMI

SEQ ID NO: 21 - protein, 192 aa, Candidatus kuenenia stuttgartiensis
NASVTLLPSDSRSIELEFTIDGFHTETLQHEGKTYQRICIQDTIQSAMPGEPQLPQCGTMVGLPVIHGVSLDILDAQYETLQGYNIYPAPKSGIEGENQDTFPSGSIKQTFFENQHIYTSNAFFPDTPVKMGNKGYLRDQPVAQVHFTPVQFNPVTGEVRIYRKIVARVSWKEGHLSPEKKTSMASPFFENL

SEQ ID NO: 22 - protein, 157 aa, Beggiatoa sp. SS
AIQIIAENDYRLTLELTLPPFEIETRQGESCQSIVMPNWAKTLKPGYPELPMTSVLIQVPPNGEITTQVIEMQDELLQNIDLCPVSYPLSVIRYQLKNDEAYQGEAFFPSALHKLEKRGILRGVPVSRLRIFPFQWHPATQALRYVTQIVFQVEFEN

SEQ ID NO: 23 - protein, 138 aa, Chloroherpeton thalassium
TFYEYKATETAVEMARAGLPVIPVKTVPVIIGTSQSPVVQVISAATQKVEGVRLAPLPKFNLEENAWDYEEGRAYSTFSFSKVAEVASVGTARGYHVAYINIYPLSYDAQTKVLKKRTRVVVSVSFSSPKKSVVKTMK

SEQ ID NO: 24 - protein, 220 aa, Desulfatibacillum alkenivorans
KTDYSKVRSLGVDDQGGGATIVTYELTGAWMDELEYRGNVYDLIDIPSAGVHTQPGHPAVPQEGLFVAIPPNAKVTAVKLLKETDRELDGKYNLIPVAEPSIEGHAEVYKPDPGIYGKDAFAPDQPFEFVGEKRVSGRLVAHILVFPAKYNPQTGKVVLVESLELEVTYDTKPGMDCAPMKRGQASNAILDSLILDADTALKMEDKLGMDADAKAAEPKK

SEQ ID NO: 25 - protein, 176 aa, Desulfococcus oleovorans
SSGMVIDLDIPGLHITETLRDGMVYHGISVPGGGRLSGIGKPSLPFVSRYVAVPQGATASVRVMDARFEEMTGYNVVPAQAPLPESNTAKGPPFEKDRVAYGENGFFPRQVAQLEGPVSIRGCETSLLQLFPVQFNPVSQTLRVYSRITVQLTFDGGTRRFIDRRKHSRSLAPVFE

SEQ ID NO: 26 - protein, 156 aa, Photobacterium profundum
FEAKLTELTLAKVKMADGNIYNRIMLPDGAAPSEPGKPNLTGYRQLVRIPDGAELELVVESVEWSKTYTDMVVDPVQLPFPDVVEEDGNRPDQHMPFVKDDAAYNALTESEVPLISVVETVRVRGKSYAVISYRPIDFNSIEGTVRFAQKVRFKVN

SEQ ID NO: 27 - protein, 127 aa, Aciduliprofundum boonei
GMRELTNPGDPAVPVKIISFTLPAGAKNIRVNLQNIWMTSYGKLKISPIPAPALKSGRAFPAKFSPPKYNEKVYKSSKYYPDKNYDFTISKTMDKTIVNVYIYPVKYNPVTNEVKVMTHAKVVVSYS

SEQ ID NO: 28 - protein, 127 aa, Aciduliprofundum boonei; identical to SEQ ID NO: 27

(19) United States
(12) Patent Application Publication
(10) Pub. No.: US 2014/0288007 A1
(43) Pub. Date: Sep. 25, 2014

(54) GINGIPAIN INHIBITORY PROPEPTIDES

(71) Applicant: Oral Health Australia Pty Ltd., Carlton, Victoria (AU)

(72) Inventors: Stuart Geoffrey Dashper, Carlton (AU); Eric Charles Reynolds, Carlton (AU); Noorjahan Laila Huq, Carlton (AU); Elena Chiew Yeen Toh, Carlton (AU)

(73) Assignee: Oral Health Australia Pty. Ltd., Carlton, Victoria (AU)

(21) Appl. No.: 14/356,125

(22) PCT Filed: Nov. 2, 2012
(86) PCT No.: PCT/AU2012/001353

§ 371 Date: May 2, 2014

(30) Foreign Application Priority Data
Nov. 4, 2011 (AU) 2011904571

(51) Int. Cl. C12N 9/64; C12Q 1/37; C07K 14/195

(52) U.S. Cl. CPC C12N 9/6472; C07K 14/195; C12Q 1/37
USPC 514/20.2; 530/324; 530/350; 514/20.1; 435/23; 435/7.4; 506/11

(57) ABSTRACT

The disclosure relates to compounds, peptides or peptidomimetics that inhibit, reduce or prevent protease activity and the use of these compounds, peptides or peptidomimetics to treat or prevent a condition. In particular the condition may be periodontal disease. The protease activity may be activity of a gingipain. The compounds, peptides or peptidomimetics of the invention may also be used in assays for the identification of protease inhibitors.

### FIELD OF THE INVENTION

The present invention relates to compounds, peptides or peptidomimetics that inhibit, reduce or prevent protease activity and the use of these compounds, peptides or peptidomimetics to treat or prevent a condition. In particular the condition may be periodontal disease. The protease activity may be activity of a gingipain. The compounds, peptides or peptidomimetics of the invention may also be used in assays for the identification of protease inhibitors.

### BACKGROUND OF THE INVENTION

Periodontal diseases are bacteria associated inflammatory diseases of the supporting tissues of the teeth and are a major public health problem. Nearly all of the human population is affected by periodontal diseases to some degree. A US Dental Health survey in 1989 reported that 85% of the studied population has periodontal diseases. The major form of periodontal disease is gingivitis which is associated with the non-specific accumulation of dental plaque at the gingival margin. The more destructive form of periodontal disease (periodontitis) is associated with a subgingivial infection by specific Gram-negative bacteria. The major bacterial pathogens implicated in this disese are known as the “red complex”, which is composed of Tannerella forsythia, Porphyromonas gingivalis and Treponema denticola. P. gingivalis is the main aetiological agent in chronic periodontitis.

The main virulence factors of P. gingivalis are its extracellular cysteine proteases, known collectively as the gingipains. Most common are RgpA and RgpB (the Arg-gingipains) and Kgp (the Lys-gingipain). The Arg-gingipains cleave proteins at the carboxyl side of Arg residues and the Lys-gingipains cleave at the carboxyl side of Lys residues.

These cell surface cysteine proteases are thought to be important for the degradation of proteins to provide peptides for growth as well as other intrinsic and extrinsic functions for survival and virulence. Several of these functions for survival and virulence may be bacterial adhesion to host tissue, hemagglutination, and the processing of bacterial cell-surface and secretory proteins. The catalytic domains of RgpA and Kgp can bind as a complex on the cell surface with a series of non-covalently bound sequence-related hemagglutinin/adhesin domains while RgpB has been shown to exist as not part of the protease adhesin complex and may consist of the catalytic domain only.

Like other cysteine proteases, the gingipains are synthesized as inactive forms with a propeptide region at the N-terminus that is removed to yield the mature, active form. The gingipains are highly conserved and the amino acid sequences of both the mature enzyme and propeptides reveal that they are only distantly related to other cysteine proteases.

There exists a need for a better or alternative inhibitor of bacterial enzymes involved in the pathogenesis of various diseases, particularly periodontal disease.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

According to the present invention there is provided a compound, peptide or peptidomimetic for inhibiting, reducing or preventing the activity of a bacterial enzyme, the compound, peptide or peptidomimetic comprising an amino acid sequence of a gingipain propeptide or fragment thereof. In one embodiment the enzyme may be an extracellular protease. Preferably, the extracellular protease is a cysteine protease, more preferably a gingipain. The protease maybe RgpA, RgpB or Kgp that is derived from a strain of Porphyromonas gingivalis.

In certain embodiments the compound, peptide or peptidomimetic is a peptide or peptidomimetic that comprises an amino acid sequence selected from the group consisting of SEQ ID NO: 1 to 10 (shown in FIG. 1).

In other embodiments the peptide or peptidomimetic comprises paraologous and orthologous sequences to those sequences shown in SEQ ID NO: 1 to 10.

In other embodiments the peptide or peptidomimetic comprises conservative subsitutions in the above amino acid sequences. These substitutions are described further below. A peptide of the invention may be isolated, purified, enriched, synthetic or recombinant.

A peptide or peptidomimetic of the invention includes an isolated, purified or recombinant amino acid sequence of a propeptide or fragment thereof as it would occur naturally when part of the cognate gingipain. In other embodiments, the peptide or peptidomimetic of the invention may include a synthetic amino acid sequence of a propeptide or fragment thereof, optionally with post-translational modifications.

In particular embodiments the peptide or peptidomimetic consists of or consists essentially of an amino acid sequence selected from the group consisting of any one of SEQ ID NOS: 1 to 10 inclusive.

In other embodiments, a peptide or peptidomimetic of the invention comprises an amino acid sequence that is at least 60, 70, 80, 90, 91, 92, 93, 94, 95, 96, 97, 98, 99 or 100% identical to an amino acid sequence selected from the group consisting of SEQ ID Nos: 1 to 28. Preferably, the group consisting of SEQ ID Nos: 1 to 10, even more preferably the group consists of SEQ ID Nos: 1 to 3.

In other embodiments, a peptide or peptidomimetic of the invention consists of or consisting essentially of an amino acid sequence that is at least 60, 70, 80, 90, 91, 92, 93, 94, 95, 96, 97, 98, 99 or 100% identical to an amino acid sequence selected from the group consisting of SEQ ID Nos: 1 to 28. In these embodiments, a compound, peptide or peptidomimetic that includes SEQ ID NOs: 1 to 28 as well as additional amino acid residues would “consist essentially of” SEQ ID NOs: 1 to 28 as long as it exhibits activity for inhibiting, reducing or preventing the activity of a bacterial enzyme, as may be determined in accordance with the assays described below. Similarly, a compound, peptide or peptidomimetic “consists essentially of” one of SEQ ID NO: 1 to 28 where it is shorter than the corresponding SEQ ID as long as it exhibits activity for inhibiting, reducing or preventing the activity of a bacterial enzyme, as may be determined in accordance with the assays described below. These embodiments thus do not include a full-length gingipain sequence. Preferably, a compound, peptide or peptidomimetic of the invention consists of or consisting essentially of an amino acid sequence that is at least 60, 70, 80, 90, 91, 92, 93, 94, 95, 96, 97, 98, 99 or 100% identical to an amino acid sequence selected from the group consisting of SEQ ID Nos: 1 to 10. Even more preferably the group consists of SEQ ID Nos: 1 to 3.

A ‘compound’ of the invention is a compound identified as an inhibitor by an assay described herein. A compound may be a protein (such as an antibody or fragment thereof or an antibody mimetic), peptide, nucleic acid (including RNA, DNA, antisense oligonucleotide, peptide nucleic acid), carbohydrate, organic compound, small molecule, natural product, library extract or from a bodily fluid.

In some embodiments, a compound, peptide or peptidomimetic of the invention has an amino acid length of between about 10 to about 300. In other embodiments, the length is between about 20 and 205 or about 50 and about 210. In other embodiments, the length is about 100 to about 200 amino acids.

Porphyromonas gingivalis is an example of gram negative bacteria that has evolved to grow under protein-rich, anaerobic conditions. The genomes of several other bacteria and archaea that exist in either protein-rich, anaerobic or more extreme conditions have recently been sequenced; some of these species have yet to be grown in vitro. These genomic studies have provided evidence for proteins that have sequence similarities with the gingipains and significant sequence similarities with the propeptides of the gingipains. Except in the case of Desulfatibacillum alkenivorans AK-01 the significant sequence similarities with the gingipain propeptide are found in the N-terminal regions as expected for propeptides.

These bacteria include: Candidatus Cloacamonas acidaminovorans, a syntrophic bacterium that is present in many anaerobic digesters; Candidatus Kuenenia stuttgartiensis, an ammonium oxidising bacteria; Chloroherpeton thalassium, a non-filamentous, flexing and gliding green sulfur bacterium that is an obligate phototroph; Desulfatibacillum alkenivorans AK-01, a mesophilic sulfate-reducer isolated from estuarine sediment that utilizes C13 to C18 alkanes, 1-alkenes (C15 and C16) and 1-alkanols (C15 and C16) as growth substrates; Desulfococcus oleovorans (strain DSM 6200/Hxd3) an alkane-degrading sulfate-reducing bacterium isolated from the saline water phase of an oil-water separator from a northern German oil field (Hxd3 is a delta-proteobacterium that is able to grow anaerobically on C12 to C20 alkanes) and Photobacterium profundum, which is classified as a piezophile, because it lives under high pressure, having been isolated at a depth of 2500 m.

Two species from the superkingdom archea have also revealed sequences that show significant similarity with the gingipain propeptide. Methanosaeta thermophila is an anaerobic thermophilic obigately aceticlastic methanogen isolated from flooded rice paddies and sewage digesters. Aciduliprofundum boonei is a cultivated obligate thermoacidophilic euryarchaeote from deep-sea hydrothermal vents.

Propeptides from these bacteria that show similarity with gingipain propeptides of SEQ ID NO: 1 to 10 are within the scope of the invention. Examples of such peptides are, but not limited to, those which have sequences of SEQ ID NO: 11 to 28 (shown in FIG. 2).

In certain embodiments there is provided a composition for inhibiting a bacterial enzyme comprising a compound, peptide or peptidomimetic of the invention and a pharmaceutically acceptable carrier. The composition can further include a divalent cation.

A composition of the invention may include propeptides with different amino acid sequences such that the composition inhibits more than one type of bacterial enzyme. For example, a composition of the invention may include two more propeptides that each exhibit selectivity for a specific gingipan, e.g. RgpA or RgpB and Kgp. In one embodiment, a composition of the invention includes propeptides having the sequence of any one or more of SEQ ID NO: 1 to 28, preferably SEQ ID NO: 1 to 10. For example, some of the propeptides in the composition may have an amino acid sequence with identity to a propeptide derived from a Kgp, while the remainder of the propeptides in the composition may have an amino acid sequence with identity to a propeptide derived from a Rgp. The level of sequence identity has already been referred to herein.

A composition of the invention includes a gingipain propeptide or fragment thereof that has been purified or enriched from a biological tissue or fluid.

In one embodiment, there is provided a method for treating or preventing one or more of the conditions described herein comprising administering to a subject an effective amount of compound, peptide, peptidomimetic or composition of the invention. In one embodiment, the compound, peptide, peptidomimetic or composition is administered directly to the gums of the subject.

In another embodiment a method of the invention further comprises administering an agent selected from the group consisting of anti-inflammatory agents, antibiotics and antibiofilm agents. The antibiotic may be selected from the group consisting of amoxicillin, doxycycline and metronidazole. Anti-inflammatory agents include Nonsteroidal Anti-inflammatory Drugs (NSAIDs). Examples of NSAIDs include compounds than inhibit a cyclooxygenase. Specific examples of NSAIDs include aspirin, ibuprofen and naproxen.

In another embodiment there is provided a method for treating or alleviating a symptom of periodontal disease in a subject, the method comprising administering to the subject a compound, peptide, peptidomimetic or composition of the invention. In another embodiment the method further includes adminstering a protein for inducing an immune response to bacteria involved in periodontal disease initiation or progresion. In one embodiment, the bacteria is P. gingivalis.

In another embodiment the invention provides a use of an effective amount of a compound, peptide, peptidomimetic or composition of the invention in the preparation of a medicament for the treatment or prevention of periodontal disease and/or the other conditions identified herein as suitable for treatment.

The present invention also provides a pharmaceutical composition for the treatment or prevention of periodontal disease (and/or the other conditions identified above as suitable for treatment) comprising an effective amount of a compound, peptide or peptidomimetic of the invention and a pharmaceutically acceptable carrier. The composition may further include an agent selected from the group consisting of anti-inflammatory agents, antibiotics and antibiofilm agents. The antibiotic may be selected from the group consisting of amoxicillin, doxycycline and metronidazole.

In another embodiment the invention provides a composition for the treatment or prevention of periodontal disease (and/or the other conditions identified above as suitable for treatment) comprising as an active ingredient a compound, peptide or peptidomimetic of the invention. The composition can further include a divalent cation.

In another embodiment the invention provides a pharmaceutical composition comprising an effective amount of a compound, peptide or peptidomimetic of the invention as a main ingredient. The composition may be used for example for the treatment or prevention of periodontal disease and/or the other conditions identified herein as suitable for treatment. Preferably, the composition further comprises a divalent cation.

In another embodiment the invention provides a compound, peptide or peptidomimetic of the invention for use in the treatment or prevention of periodontal disease and/or the other conditions identified herein as suitable for treatment.

In another embodiment the invention provides a composition comprising a compound, peptide or peptidomimetic of the invention for use in the treatment or prevention of periodontal disease. Preferably, the composition further comprises a divalent cation.

The divalent cation is preferably selected from the group consisting of Zn²⁺, Ca²⁺, Cu²⁺, Ni²⁺, Co²⁺, Fe²⁺, Sn²⁺, and Mn²⁺. In addition, the divalent cation may be in association with fluoride such as SnF⁺ and CuF⁺. It is currently preferred, however, that the divalent cation is Ca²⁺ or Zn²⁺.

It is further preferred that the ratio of the divalent cation to the peptide is in the range of 1.0:2.0 to 1.0:10.0, preferably in the range of 1.0:4.0.

The present invention also provides an assay for identifying an inhibitor of a cysteine protease comprising the steps of:
contacting a cysteine protease with a candidate compound in the presence of a compound, peptide or peptidomimetic of the invention,
determining whether the candidate compound competes with the compound, peptide or peptidomimetic of the invention;

wherein competition indicates that the candidate compound is an inhibitor of a cysteine protease.

The present invention also provides an assay for identifying an inhibitor of a cysteine protease comprising the steps of:
contacting a cysteine protease with a compound, peptide or peptidomimetic of the invention in the presence or absence of a candidate compound,
determining the level of the compound, peptide or peptidomimetic bound to the protease,

wherein a reduction in the level of the compound, peptide or peptidomimetic in the presence of the candidate compound compared to the absence of the candidate compound thereby identifies the candidate compound as an inhibitor of a cysteine protease.

The present invention also provides an assay for identifying an inhibitor of a cysteine protease comprising the steps of:
contacting a cysteine protease with a candidate compound in the presence or absence of a compound, peptide or peptidomimetic of the invention,
determining the level of the candidate compound bound to the protease,

wherein a reduction in the level of the candidate compound in the presence of the compound, peptide or peptidomimetic compared to the absence of the compound, peptide or peptidomimetic thereby identifies the candidate compound as an inhibitor of a cysteine protease.

The present invention also provides an assay for identifying an inhibitor of a cysteine protease comprising the steps of:
providing a compound, peptide or peptidomimetic of the invention, in the presence or absence of a candidate compound, in conditions that allow binding of the compound, peptide or peptidomimetic of the invention to a cysteine protease,
determining the level of the compound, peptide or peptidomimetic bound to the protease,

wherein a reduction in the level of the compound, peptide or peptidomimetic in the presence of the candidate compound compared to the absence of the candidate compound thereby identifies the candidate compound as an inhibitor of a cysteine protease.

The present invention also provides an assay for identifying an inhibitor of a cysteine protease comprising the steps of:
providing a candidate compound, in the presence or absence of a compound, peptide or peptidomimetic of the invention, in conditions that allow binding of the candidate compound to a cysteine protease,
determining the level of the candidate compound bound to the protease,

wherein a reduction in the level of the candidate compound in the presence of the compound, peptide or peptidomimetic compared to the absence of the compound, peptide or peptidomimetic thereby identifies the candidate compound as an inhibitor of a cysteine protease.

Preferably, the candidate compound identified as an inhibitor of a cysteine protease is assayed one or more times in accordance with the steps described herein with a further cysteine protease and the same or a further compound, peptide or peptidomimetic of the invention to determine whether the candidate compound inhibits one or more cysteine proteases.

Preferably the candidate compound is an antibody or fragment thereof, or an antibody mimetic such as an anticalin. The candidate compound may be part of a library in which case the assay is performed in high-throughput.

Preferably, the cysteine protease is a gingipain, more preferably Kgp, RgpA or RgpB. Even more preferably the gingpain is Kgp.

A compound, peptide or peptidomimetic of the invention useful in an assay of the invention has already been defined herein. Preferably, the compound, peptide or peptidomimetic of the invention comprises an amino acid sequence that is at least 60, 70, 80, 90, 91, 92, 93, 94, 95, 96, 97, 98, 99 or 100% identical to an amino acid sequence selected from the group consisting of SEQ ID Nos: 1 to 28. Preferably, the group consisting of SEQ ID Nos: 1 to 10, even more preferably the group consists of SEQ ID Nos: 1 to 3. In other embodiments, a compound, peptide or peptidomimetic of the invention consists of or consisting essentially of an amino acid sequence that is at least 60, 70, 80, 90, 91, 92, 93, 94, 95, 96, 97, 98, 99 or 100% identical to an amino acid sequence selected from the group consisting of SEQ ID Nos: 1 to 28.

In one embodiment, the invention provides a compound, peptide or peptidomimetic of the invention for use in an assay of the invention. In one embodiment, the invention provides a compound, peptide or peptidomimetic of the invention when used in an assay of the invention. In one embodiment, the invention provides a compound, peptide or peptidomimetic labelled for use in an assay of the invention.

The invention also provides a recombinant or synthetic protein consisting of or consisting essentially of an amino acid sequence of the catalytic domain of Kgp. In other words, the protein consisting of an amino acid sequence of the catalytic domain of Kgp is not linked or does not interact with an adhesin domain.

In one embodiment, the invention provides a use of the recombinant or synthetic protein consisting of or consisting essentially of an amino acid sequence of the catalytic domain of Kgp or Rgp in an assay of the invention to identify an inhibitor of a cysteine protease, preferably Kgp or Rgp. In one embodiment, the invention provides the recombinant or synthetic protein consisting of or consisting essentially of an amino acid sequence of the catalytic domain of Kgp or Rgp when used in an assay of the invention to identify an inhibitor of a cysteine protease, preferably Kgp or Rgp.

The present invention also provides use of a compound identified by an assay described herein to inhibit a cysteine protease. Preferably, the cysteine protease is Kgp or Rgp. Even more preferably the cysteine protease is Kgp. In one embodiment the invention also provides use of a compound identified as an inhibitor by an assay described herein to treat or prevent periodontal disease.

The invention also provides a method of treating or preventing periodontal disease and/or the other conditions identified herein as suitable for treatment or prevention, including administering a peptide or peptidomimetic of the invention and/or a compound identified by an assay as described herein as an inhibitor of a cysteine protease.

As it is the physical nature of the peptides rather than the specific sequence of the peptide which results in their protease inhibitory activity so called conservative substitutions may be made in the peptide sequence with no substantial loss of activity. It is intended that such conservative substitutions which do not result in a substantial loss of activity are encompassed in the present invention.

Whilst the concept of conservative substitution referred to above is well understood by the person skilled in the art, for the sake of clarity conservative substitutions are those set out below.
Gly, Ala, Val, Ile, Leu, Met;
Asp, Glu;
Asn, Gln;
Ala, Ser, Thr;
Lys, Arg, His;
Phe, Tyr, Trp, His; and
Pro, Nα-alkalamino acids.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

FIG. 1: Amino acid sequences of gingipain propeptides from various strains of Porphryomonas gingivalis.

FIG. 2: Amino acid sequences of propeptides from bacteria other than Porphryomonas gingivalis.

FIG. 3: Ion exchange chromatography of the desalted, acetone-precipitated proteins from the P. gingivalis Kgp_{cat}ΔABM1 mutant ECR368 culture supernatant, using a Q-Sepharose column attached to an AKTA-Basic FPLC system. The column was eluted with 10 mM Sodium acetate pH 5.3 and then a linear gradient of 0-1 M NaCl in 10 mM sodium acetate was applied. The eluant was monitored at an absorbance of 280 nm. The collected fractions were measured for Lys- and Arg-specific proteolytic activity. The fractions containing Lys-activity were pooled and collected for further purification.

FIG. 4: Gel filtration purification of the concentrated samples of ECR368 culture supernatant after desalting using a Superdex G75 column attached to an AKTA-Basic FPLC system. The column was eluted with TC50 buffer, pH 8.0, 1.0 mL/min. The eluant was monitored at an absorbance of 280 and 215 nm. Fractions A8-A9 contains active KgpcatΔABM1.

FIG. 5: SDS-PAGE of Kgp_{cat}ΔABM1 enriched fraction from P. gingivalis ECR368. Lanes contain; lane 1: See-Blue® Pre-Stained standard, where sizes in kDa are indicated, lane 2: culture supernatant, lane 3: culture supernatant after acetone precipitation, lane 4: culture supernatant after acetone precipitation and ultracentrifugation, lane 5: Kgp_{cat}ΔABM1 enriched fraction after gel filtration purification. The gel was Coomassie® stained.

FIG. 6: (A) Gel filtration chromatography of the rKgp propeptide using Superdex G75 column equilibrated with 50 mM NH₄HCO₃ attached to an AKTA-Basic FPLC system. The eluant was monitored at an absorbance of 280 and 215 nm. (B) MALDI-TOF MS analysis of the rKgp propeptide with the His-tag still attached showed a singly-charged (m/z 25,446.9 [M⁺H]⁺), a dual-charged (m/z 12728.8 [M⁺2H]²⁺) and a triply charged (m/z 8486.5 [M⁺3H]³⁺) signal, each corresponding to the target molecular mass (25,285 Da).

FIG. 7: Kgp_{cat}ΔABM1 enriched fraction proteolytic activity (Units/mg) with 20.0 and 40.0 mg/L rKgp propeptide (rKgpPro) at 1 mM cysteine in the assay with the chromogenic GPKNa substrate. The final concentration of Kgp_{cat}ΔABM1 enriched fraction per well is 1.16 mg/L. All samples were significantly different (p<0.05) from the control.

FIG. 8: Proteolytic assay using chromogenic substrate GPKNa confirming that the rate of substrate hydrolysis was linear throughout the assay. Kgp_{cat}ΔABM1 enriched fraction proteolytic activity (Units/mg) with 0 mg/L ( [symbol] ) and 40.0 mg/L rKgp propeptide (rKgpPro) ( [symbol] ) at 1 mM cysteine in the assay with the chromogenic GPKNa substrate. The final concentration of Kgp_{cat}ΔABM1 enriched fraction per well was 1.16 mg/L. The rate of substrate hydrolysis was linear throughout the assay.

FIG. 9: RP-HPLC profile of the chromogenic assay (GPKNa) post-incubation mixtures applied to an analytical RP-HPLC column (C18) and eluted using a linear gradient of 0-100% buffer B in 30 min at a flow rate of 1.0 mL/min. The eluant was detected at 214 nm. (A) Incubation mixture of the Kgp_{cat}ΔABM1 enriched fraction without propeptide (B) Incubation mixture of the Kgp_{cat}ΔABM1 enriched fraction and rKgp propeptide.

FIG. 10: Analysis of Lys-specific chromogenic assay (GPK-NA) products by SDS-PAGE. The following assay contents were electrophoresed: KgpcatΔABM1 enriched fraction with rKgp propeptide (rKgpPro) (lane 2). Lane 1 shows the molecular weight (MW) markers (See-Blue® Pre-Stained standard, lane 1), labelled in kDa. The gel was Coomassie® stained.

FIG. 11: A secondary plot for the estimation of inhibition constant (Ki′) of Kgp_{cat}ΔABM1 enriched fraction by rKgp propeptide. The Vₘₐₓ observed values were plotted against the inhibitor concentration. The Ki′ for Kgp propeptide was calculated to be 2.01 μM.

FIG. 12: Kgp proteolytic activity measured using fluorescent BSA substrate (DQ™ BSA) with 1, 5, and 10 mg/L rKgp propeptide (rKgpPro). The final concentration of Kgp per well is 1.16 mg/L. The fluorescence value for the negative control (TLCK 1 mM-treated proteases) was subtracted from each value. The error bars were calculated as a standard deviation of 3-6 replicates. All samples were significantly different (p<0.05) from the control.

FIG. 13: Analysis of fluorescent BSA assay products by SDS-PAGE. The gels were Coomassie® stained. (A) The following assay contents were electrophoresed: Kgp_{cat}ΔABM1 enriched fraction (from control wells, lanes 2-3), Kgp_{cat}ΔABM1 enriched fraction with rKgp propeptide (rKgpPro) (lanes 4-5). Lane 1 shows the molecular weight (MW) markers (See-Blue® Pre-Stained standard, lane 1), labelled in kDa. (B) Samples from the assay were electrophoresed as follows; Kgp_{cat}ΔABM1 enriched fraction (Kgp) (from control wells, lanes 2-3), Kgp_{cat}ΔABM1 enriched fraction with rKgp propeptide (rKgpPro) (lanes 4-5), Kgp_{cat}ΔABM1 enriched fraction with TLCK (lanes 6-7). MW indicates molecular markers (See-Blue® Pre-Stained standard, lane 1), where sizes in kDa are indicated.

FIG. 14: Time course of RgpB proteolytic activity using the DQ-BSA fluorescent substrate. Fluorescence was measured over 11 hours at 37° C. with a reading taken every hour.

FIG. 15: RgpB proteolytic activity measured using fluorescent BSA substrate (DQ™ BSA) with 0.1, 1, 5, 10 mg/L rRgp propeptide (rRgp Pro). The final concentration of RgpB per well is 1.16 mg/L. The fluorescence value for the negative control (TLCK 1 mM-treated proteases) was subtracted from each value. The error bars were calculated as a standard deviation of 3-6 replicates. All samples were significantly different (p<0.05) from the control except the values for 0.1 mg/L and different from other values except between the values for 5 and 10 mg/L.

FIG. 16: A secondary plot for the estimation of inhibition constant (Ki′) of RgpB enriched fraction by RgpB propeptide. The Vₘₐₓ observed values were plotted against the inhibitor concentration. The Ki′ for RgpB propeptide was calculated to be 11.8 nM.

FIG. 17: Relative growth of P. gingivalis in a protein-based minimal medium in the presence of rRgpB propeptide (R-pp) and/or Kgp propeptide (K-pp).

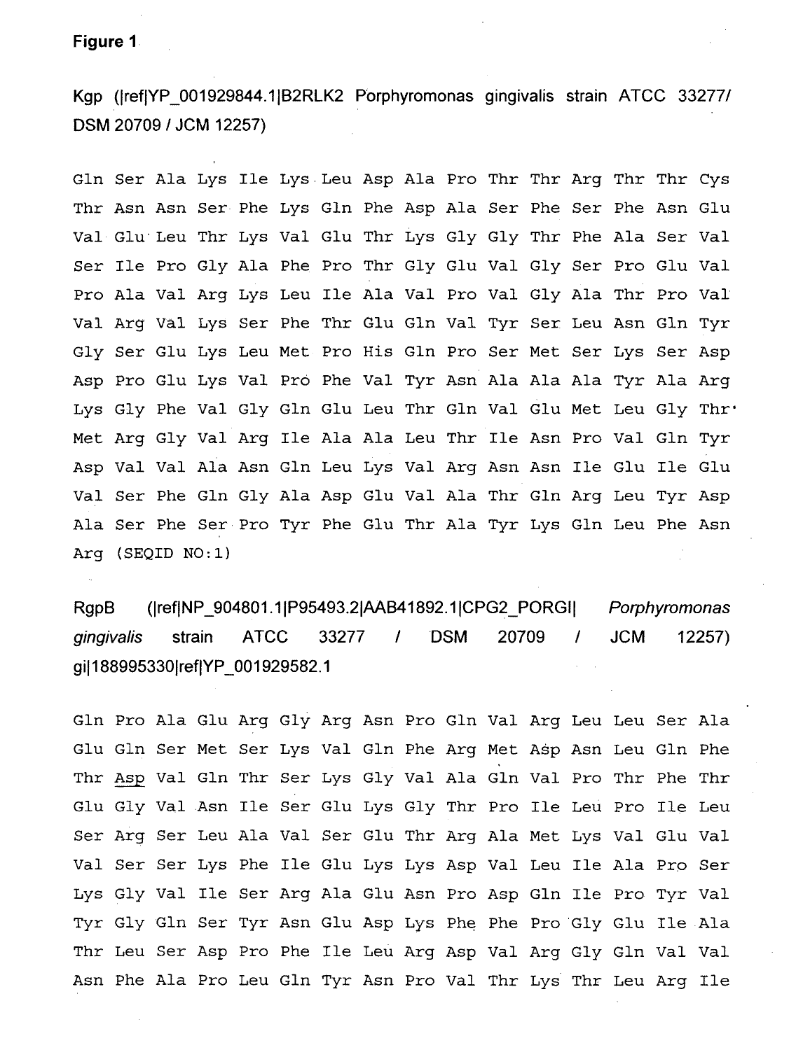

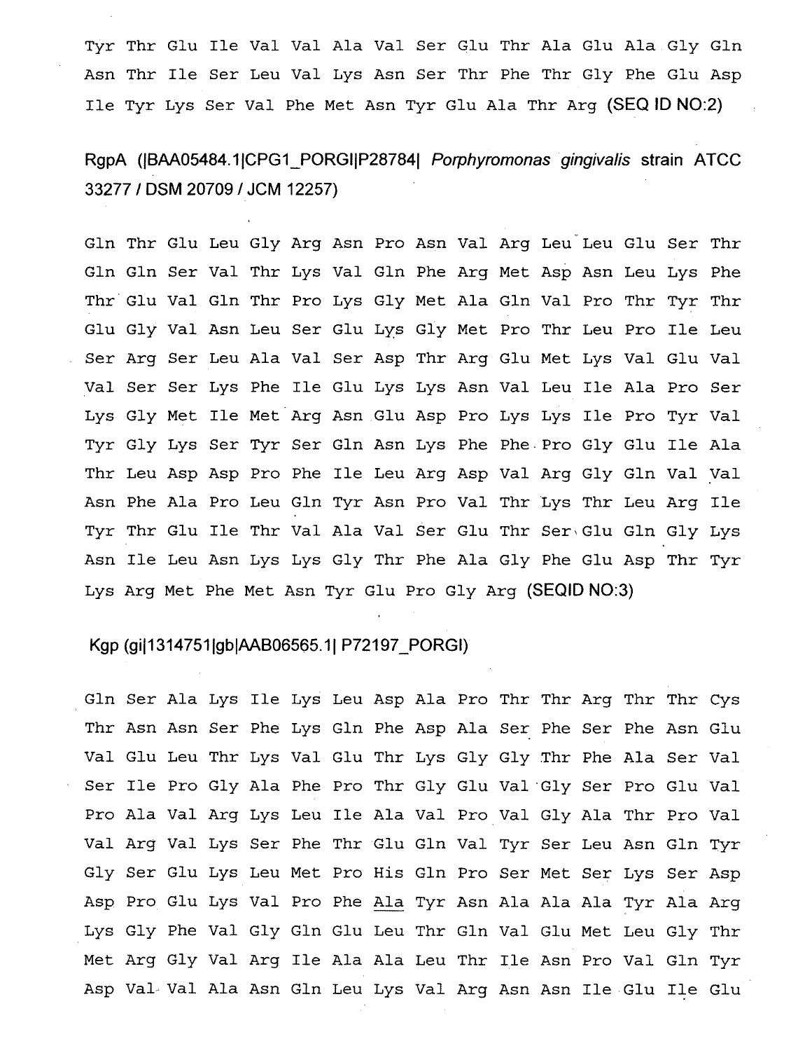

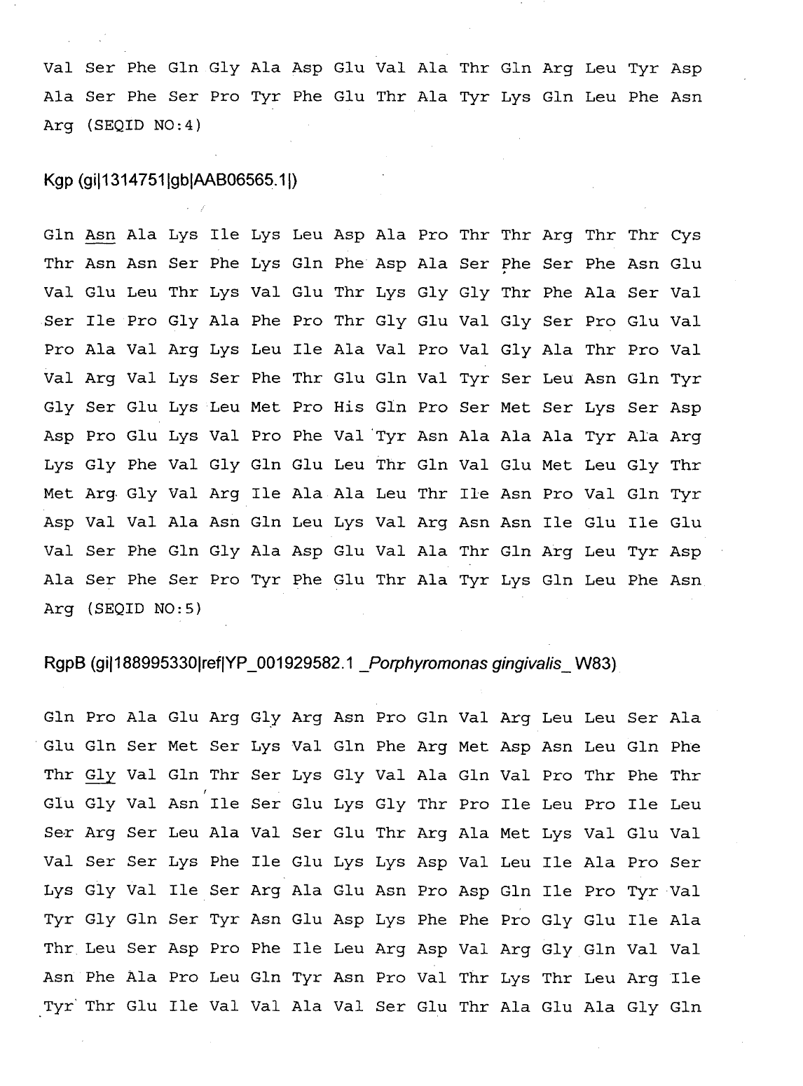

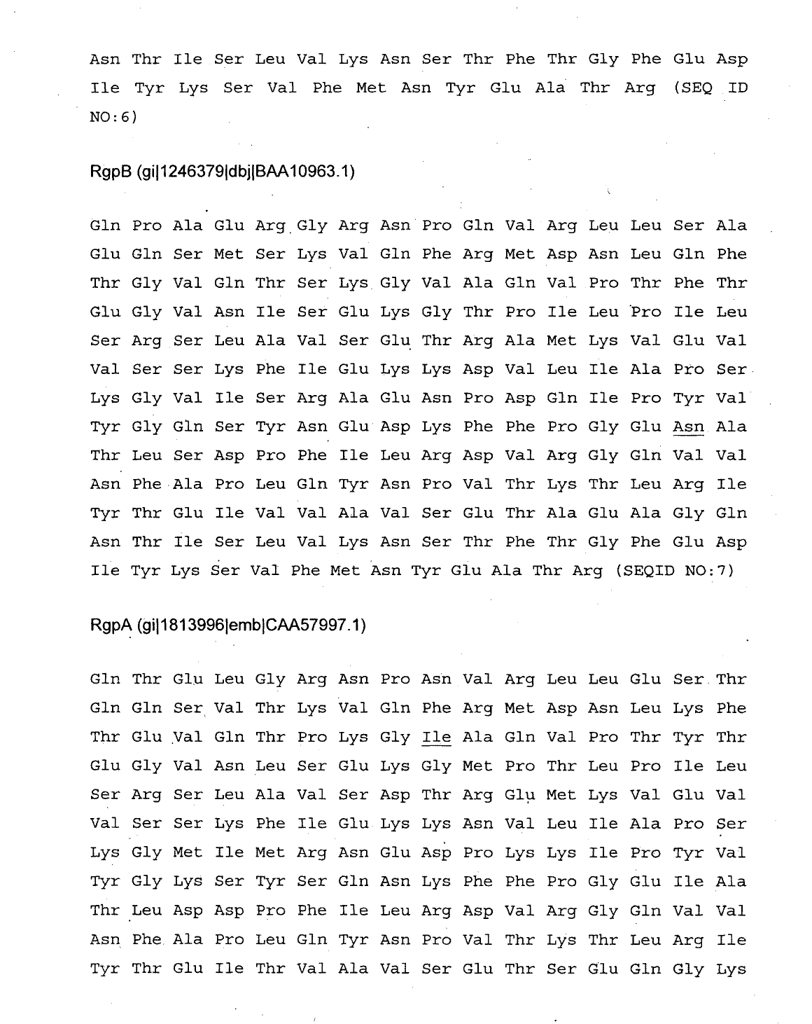

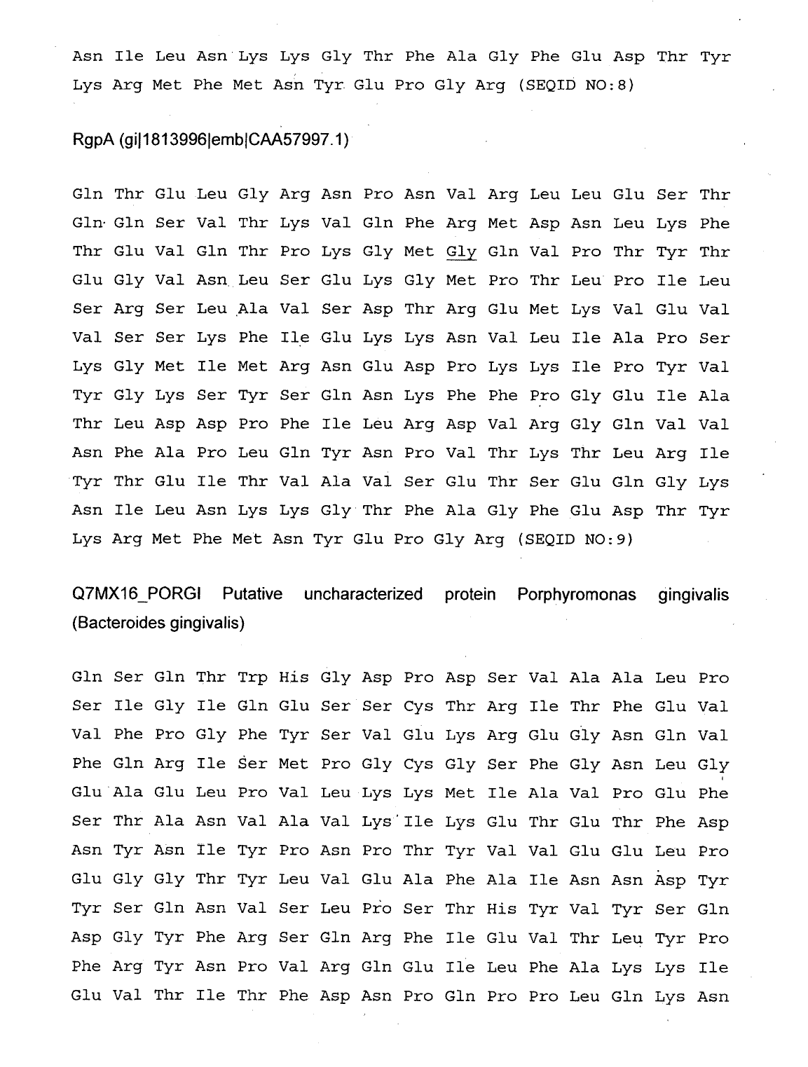

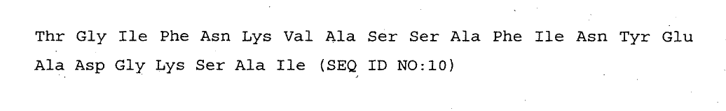

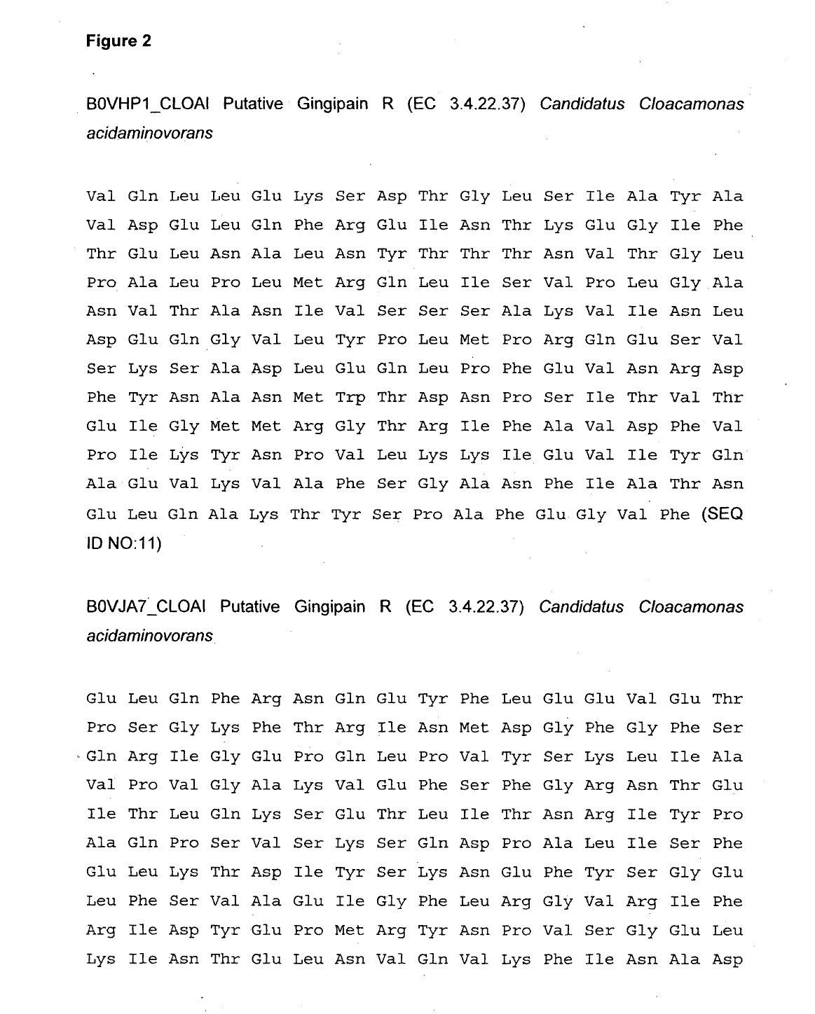

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All of the patents and publications referred to herein are incorporated by reference in their entirety. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

Compounds, peptide or peptidomimetics that exhibit protease inhibitory activity have the potential to be developed in the area of oral care, functional foods, and pharmaceuticals. The present invention includes peptides that are characterized by the ability to inhibit extracellular protease activity. These peptides may be produced synthetically or expressed recombinantly. These peptides have several advantages including, but not limited to, that they are non-toxic, biocompatible and are derived from the cognate zymogen.

As used herein, a “propeptide” is a sequence of amino acids N-terminal to the catalytic domain which when cleaved from the gingipain, such that it is no longer linked to the gingipain, results in the marked increase in catalytic activity of the gingipain.

The invention also includes functional fragments of the amino acid sequences of SEQ ID NO: 1 to 28. A functional fragment is an amino acid sequence that is shorter than the amino acid sequences corresponding to SEQ ID NO: 1 to 28 but still retains the function of the corresponding amino acid sequences to SEQ ID NO: 1 to 28. A functional fragment can be easily determined by shortening the amino acid sequence, for example using an exopeptidase, or by sythesizing amino acid sequences of shorter length, and then testing for any protease inhibitory activity.

Also within the scope of the invention are variants of the amino acid sequences of SEQ ID NO: 1 to 3 corresponding to orthologous or paralogous sequences. Examples of such sequences include those shown in SEQ ID NOS 4 to 28.

It will be understood by a person skilled in the art that one or more amino acid deletions to the amino acid sequence defined by any one of SEQ ID Nos: 1 to 28 may be made without losing the capacity of the compound, peptide or peptidomimetic to inhibit, reduce or prevent protease activity. Experiments, including those described herein, can be performed to determined whether a compound, peptide or peptidomimetic that has an amino acid sequence that differs to any one of SEQ ID Nos: 1 to 28 by one or more amino acid deletions can still inhibit, reduce or prevent protease activity.

The compound, peptide, peptidomimetic or composition of the invention may be administered directly to the gums of the subject in need of treatment or prevention of periodontal disease. Topical administration of the composition of the invention is preferred, however it will be appreciated by a person skilled in the art that a compound, peptide, peptidomimetic or composition may also be administered parenterally, e. g, by injection intravenously, intraperitoneally, intramuscularly, intrathecally or subcutaneously.

In one embodiment the compound, peptide or peptidomimetic may be a part of a composition applicable to the mouth such as dentifrice including toothpastes, toothpowders and liquid dentifrices, mouthwashes, troches, chewing gums, dental pastes, gingival massage creams, gargle tablets, dairy products and other foodstuffs.

Alternatively, the compound, peptide, peptidomimetic of the invention may be formulated as a composition for oral administration (including sublingual and buccal), pulmonary administration (intranasal and inhalation), transdermal administration, and rectal administration.

Inhibition of an enzyme may be competitive or non-competitive. Without wishing to be bound by any theory or mode of action, it is believed that propeptides are non-competitive inhibitors that do not compete with substrate for binding to the catalytic site of a target enzyme. It is believed that propeptides binds to the enzyme at a site other than the catalytic site.

A composition of the invention may include a peptide or peptidomimetic of the invention and a compound identified as an inhibitor of the catalytic site of a bacterial enzyme, such as a cysteine protease. Preferably, the cysteine protease is a gingipain, such as a Kgp or Rgp. In one embodiment, the composition includes a peptide or peptidomimetic of the invention and a compound idenitified as a competitive inhibitor of the same enzyme which the peptide or peptidomimetic of the invention inhibits.

Although the invention finds application in humans, the invention is also useful for veterinary purposes. The invention is useful for domestic or farm animals such as cattle, sheep, horses and poultry; for companion animals such as cats and dogs; and for zoo animals.

A subject in need of treatment may be one which exhibits subclinical or clinical symptoms of periodontal disease. Subclinical or clinical manifestations of periodontal disease include acute or chronic inflammation of the gingiva. The hallmarks of acute inflammation may be present including an increased movement of plasma and leukocytes from the blood into the injured tissues. Clinical signs of acute infection of the gingiva may also be present including rubor (redness), calor (increased heat), tumor (swelling), dolor (pain), and functio laesa (loss of function). Chronic inflammation may be characterised by leukocyte cell (monocytes, macrophages, lymphocytes, plasma cells) infiltration. Tissue and bone loss may be observed. A subject in need of treatment may also be characterised by having an increased level of P. gingivalis bacteria present at a periodontal site, above a normal range observed in individuals without periodontal disease.

The route of administration may depend on a number of factors including the nature of the compound, peptide, peptidomimetic or composition to be administered and the severity of the subject's condition. It is understood that the frequency of administration of a compound, peptide, peptidomimetic or composition of the invention and the amount of compound, peptide, peptidomimetic or composition of the invention administered may be varied from subject to subject depending on, amongst other things, the stage of periodontal disease initiation or progression in the subject. The frequency of administration may be determined by a clinician.

It is also contemplated that any disease, condition or syndrome that is a consequence of or associated with protease activity of a gingipain or related protease, may be prevented or treated by a compound, peptide, peptidomimetic or composition of the invention. In addition, a symptom of a disease, condition or syndrome that is a consequence of or associated with protease activity of a gingipain or related protease, may be reduced in severity or incidence by a compound, peptide, peptidomimetic or composition of the invention. Further more, other diseases, conditions or syndromes that are a consequence of or associated with periodontal disease may also be treated or the risk of developing these diseases, conditions or syndromes may be reduced. For example, periodontal disease may increase the risk of an individual developing cardiovascular disease. This increase risk of developing cardiovascular disease may be reduced by treating periodontal disease by administering a compound, peptide, peptidomimetic or composition of the invention to an individual with periodontal disease.

A representative assay to identify an inhibitor of a cysteine protease is a “competitive binding assay” or “competition binding assay”. Competitive binding assays are serological assays in which unknowns (e.g. candidate compounds) are detected and quantitated by their ability to inhibit the binding of a labeled known compound to its specific target. The labelled known compound used herein may be a compound, peptide or peptidomimetic of the invention which when employed in such immunoassays may be labeled or unlabeled. A labeled compound, peptide or peptidomimetic may be employed in a wide variety of assays, employing a wide variety of labels. Detection of the formation of a compound-target complex between a compound, peptide or peptidomimetic of the invention and a cysteine protease can be facilitated by attaching a detectable substance to the compound, peptide or peptidomimetic. Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Such labeled reagents may be used in a variety of well-known assays, such as radioimmunoassays, enzyme immunoassays, e.g., ELISA, fluorescent immunoassays, and the like. See, for example, U.S. Pat. Nos. 3,766,162; 3,791,932; 3,817,837; and 4,233,402.

Competition assays are known in the art. Competitive assays are widely used for different purposes such as agonist/antagonist interactions with a receptor or for concentration analysis for a drug of interest. In one example, an affinity-purified capture antibody pre-coated onto a microplate is used, to which a limited concentration of enzyme-linked analyte along with the non-labeled sample analyte are added simultaneously. Both analytes will then compete for the limited number of binding sites on the primary antibody. Substrate is added and hydrolyzed by the enzyme, thereby producing a color product that can be measured (exactly like an ELISA). The amount of labeled analyte bound is inversely proportional to the amount of unlabeled analyte presenting the sample (signal decreases as analyte concentration increases).

The candidate compound can be any compound which one wishes to test including, but not limited to, proteins (such as antibodies or fragments thereof or antibody mimetics), peptides, nucleic acids (including RNA, DNA, antisense oligonucleotide, peptide nucleic acids), carbohydrates, organic compounds, small molecules, natural products, library extracts, bodily fluids. The candidate compound may be part of a library, for example a collection of compounds containing variations or modifications.

Non-limiting examples of antibody mimetics or alternate immunoglobulin molecules include those described by Dimitrov, 2009, MAbs 1 26-28; whilst examples of non-immunoglobulin protein scaffolds are described in Skerra, 2007 Current Opinions in Biotechnology, 18 295-304.

Anticalins are proteins that are not structurally related to antibodies but are a class of antibody mimetics. Anticalins are derived from human lipocalins which are a family of binding proteins. Anticalins are about eight times smaller than antibodies with a size of about 180 amino acids and a mass of about 20 kDa.

Anticalins have better tissue penetration than antibodies and are stable at temperatures up to 70° C. Unlike antibodies, they can be produced in bacterial cells like E. coli in large amounts.

The assay methods of the invention include high-throughput screening applications. For example, a high-throughput screening assay may be used which comprises any of the assays according to the invention wherein aliquots of cysteine proteases are exposed to a plurality of candidate compounds within different wells of a multi-well plate. Further, a high-throughput screening assay according to the disclosure involves aliquots of cysteine protease which are exposed to a plurality of candidate compounds in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention.

Prior to the present invention, there was no assay available which reliably identified an inhibitor of a cysteine protease. The work of the present inventors led to the production of isolated gingipain propeptides that retain the ability to inhibit gingipain activity. These propeptides can be used in assays to direct the identification of compounds that not only bind to a cysteine protease but also inhibit cysteine protease activity. These identified inhibitors of cysteine protease activity can then be used in a clinical setting to treat diseases that are involved with the activity of a cysteine protease. Alternatively, the identified inhibitor may be subject to optimisation such that its affinity and/or inhibitory activity for a cysteine protease is increased.

The assay of the invention also allows the identification of inhibitors that have inhibitory activity towards a specific type of cysteine protease. A candidate compound may be assayed repeatedly in the presence of different cysteine proteases to determine whether the candidate compound inhibits only one type of cysteine protease or have inhibitory activity towards more than one type of cysteine protease. For example, either a Kgp or a Kgp-like gingipain, or alternatively, inhibit a Rgp or Rgp-like gingipain.

The concentration of labeled compound, peptide or peptidomimetic of the invention bound to the cysteine protease is inversely proportional to the ability of the candidate compound to compete in the binding assay. Conversely, if the candidiate compound is labelled then the ability of a compound, peptide or peptidomimetic of the invention to compete in the binding assay indicates that the candidate compound binds to a simlar region of the cysteine protease as a gingipain propeptide.

A variety of other reagents may also be included in the screening assay. These include reagents like salts, neutral proteins, e.g., albumin, detergents, etc. that are used to facilitate optimal protein-protein binding and/or reduce non-specific or background interactions. Reagents that improve the efficiency of the assay, such as protease inhibitors, nuclease inhibitors, antimicrobial agents, etc., may be used. The mixture of components are added in any order that provides for the requisite binding. Incubations are performed at any suitable temperature, typically between about 0 and about 40° C., preferably, 20, 25, 26, 27, 28, 29, 30, 31, 32, 33, 34, 35, 36 or 37° C. Incubation periods are selected from about 0.05 to about 10 hours. Preferably the incubation period allows the molecular interactions occurring within the assay to reach equilibrium.

The present invention also provides methods for making a peptide of the invention. In one preferred embodiment, the method comprises the following steps:

(1) expressing a nucleic acid which encodes for a peptide according to any one of SEQ ID No: 1 to 28, in an appropriate prokaryotic or eukaryotic expression system; and

(2) isolating or purifying the expressed peptide.

Preferably, the method further includes the preceding step of generating a nucleic acid which encodes for a peptide of the invention (for example any one of SEQ ID No: 1 to 28), the nucleic acid being modified so as to inactivate a known or predicted cleavage site in the peptide. Lysine and arginine residues are the expected autocleavage sites during the processing of the mature gingipains, hence amino acid sequences and nucleic sequences that encode them which have these or other cleavage sites replaced by amino acids which inhibit or reduce cleavage, for example glutamines and asparagines, are within the scope of the invention.

Expression systems are well known in the molecular biology art as are methods for isolation and purification of expressed proteins.

The nucleic acid molecule encoding a peptide of the invention may, for example, be inserted into a suitable expression vector for production of the peptide by insertion of the expression vector into a prokaryotic or eukaryotic host cell. Successful expression of the recombinant peptide requires that the expression vector contains the necessary regulatory elements for transcription and translation which are compatible with, and recognised by the particular host cell system used for expression. A variety of host cell systems may be utilized to express the recombinant protein, which include, but are not limited to bacteria transformed with a bacteriophage vector, plasmid vector, or cosmid DNA; yeast containing yeast vectors; fungi containing fungal vectors; insect cell lines infected with virus (e.g. baculovirus); and mammalian cell lines transfected with plasmid or viral expression vectors, or infected with recombinant virus (e.g. vaccinia virus, adenovirus, adeno-associated virus, retrovirus, etc).

Using methods known in the art of molecular biology, various promoters and enhancers can be incorporated into the expression vector, to increase the expression of the recombinant peptide, provided that the increased expression of the amino acid sequences is compatible with (for example, non-toxic to) the particular host cell system used.

The selection of the promoter will depend on the expression system used. Promoters vary in strength, i.e. ability to facilitate transcription. Generally, it is desirable to use a strong promoter in order to obtain a high level of transcription of the coding nucleotide sequence and expression into recombinant protein. For example, bacterial, phage, or plasmid promoters known in the art from which a high level of transcription have been observed in a host cell system including E. coli include the lac promoter, trp promoter, recA promoter, ribosomal RNA promoter, the P_{R} and P_{L} promoters, lacUV5, ompF, bla, Ipp, and the like, may be used to provide transcription of the inserted nucleotide sequence encoding amino acid sequences.

Other control elements for efficient transcription or translation include enhancers, and regulatory signals. Enhancer sequences are DNA elements that appear to increase transcriptional efficiency in a manner relatively independent of their position and orientation with respect to a nearby coding nucleotide sequence. Thus, depending on the host cell expression vector system used, an enhancer may be placed either upstream or downstream from the inserted coding sequences to increase transcriptional efficiency. Other regulatory sites, such as transcription or translation initiation signals, can be used to regulate the expression of the coding sequence.

In another embodiment, the expression plasmids may optionally contain tags allowing for convenient isolation and/or purification of the expressed proteins. The use of expression plasmids and the methods for isolating and purifying the tagged protein products are well known in the art.

Peptides of the invention can be produced by a variety of known techniques. For example such peptides or fragments therof can be synthesized (eg chemically or recombinantly), isolated, purified and tested for their ability to form complexes with mature gingipains using methods described herein or methods known in the art. Alternatively, peptides or fragments therof may be recombinantly produced using various expression systems (eg E. coli, Chinese Hamster Ovary cells, COS cells baculovirus) as is well known in the art. A peptide of the invention may also be produced by digestion of naturally occurring or recombinantly produced gingipain propeptide or gingipain precursors using for example a protease (eg trypsin, chymotrypsin). Computer analysis can be used to identify proteolytic cleavage sites. Alternatively peptides may be produced from naturally occurring or recombinantly produced gingipain propeptide or gingipain precursors using such standard techniques in the art as by chemical cleavage (eg cyanogen bromide, hydroxylamine, formic acid).

A compound, peptide or peptidomimetic of the invention may comprise as many amino acids as are necessary to bind to the target protease, thereby inhibiting partially or completely protease activity. In one embodiment, the target protease in a gingipain and partial or complete inhibition of gingipain activity can be demonstrated in assays involving P. gingivalis whole cells or harvested outer membrane complex or purified gingipains.

A compound, peptide or peptidomimetic having a sequence of SEQ ID NO: 1 to 28 may also have point mutations or other modifications introduced (including insertion, deletion and substitution) to improve a biochemical property, for example to enhance the activity or circulatory or storage half-life. In addition, as discussed further herein point mutations may be introduced into one or more proteolytic cleavage sites to prevent or inhibit proteolytic degradation of the compound, peptide or peptidomimetic in vivo. All variants discussed herein are within the scope of the invention provided such variants maintain the ability to inhibit, reduce or prevent the activity of a bacterial enzyme.

Accordingly, nucleic acids of the invention include, in addition to those encoding SEQ ID NO: 1 to 28, nucleic acids which differ in nucleotide sequence by allelic variations (naturally-occurring base changes in the species population which may or may not result in an amino acid change). The invention also includes nucleic acid sequence caused by point mutations or by induced modifications (e.g., insertion, deletion, and substitution) to enhance the activity, half-life or production of the gingipain propeptides encoded are also useful for the present invention. Computer programs that are used to determine DNA sequence homology are known in the art.

A ‘peptidomimetic’ is a synthetic chemical compound that has substantially the same structure and/or functional characteristics of a peptide of the invention, the latter being described further herein. Typically, a peptidomimetic has the same or similar structure as a peptide of the invention, for example the same or similar sequence of SEQ ID NO: 1 to 28 or fragment thereof. A peptidomimetic generally contains at least one residue that is not naturally synthesised. Non-natural components of peptidomimetic compounds may be according to one or more of: a) residue linkage groups other than the natural amide bond (‘peptide bond’) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

Peptidomimetics can be synthesized using a variety of procedures and methodologies described in the scientific and patent literatures, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

The compounds, peptides or peptidomimetics of the invention can be administered in the form of a pharmaceutical composition. These compositions may be manufactured under GMP conditions or in some embodiments by means of conventional mixing, dissolving, granulating, dragee-making, levigating, emulsifying, encapsulating, entrapping or lyophilizing processes.

Pharmaceutical compositions may be formulated using one or more physiologically acceptable carriers, diluents, excipients or auxiliaries. The ingredients may facilitate processing peptides or peptidomimetics into preparations which can be used pharmaceutically.

Administration for treatment can be parenteral, intravenous, oral, subcutaneous, intraarterial, intracranial, intrathecal, intraperitoneal, topical, intranasal or intramuscular.

Pharmaceutical compositions for parenteral administration are generally sterile and substantially isotonic. Physiologically compatible buffers such as Hank's solution, Ringer's solution, or physiological saline or acetate buffer may be used. The solution may also contain suspending, stabilizing and/or dispersing agents. The peptides or peptidomimetics may be provided in powder form to be dissolved in solvent such as sterile pyrogen-free water, before use.

“Percent (%) amino acid sequence identity” or “percent (%) identical” with respect to a peptide or polypeptide sequence, i.e. a peptide of the invention defined herein, is defined as the percentage of amino acid residues in a candidate sequence that are identical with the amino acid residues in the specific peptide or polypeptide sequence, i.e. a peptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM 120 weight residue table, a gap length penalty of 12, and a gap penalty of 4 can be used.

An oral composition of this invention which contains the above-mentioned pharmaceutical composition can be prepared and used in various forms applicable to the mouth such as dentifrice including toothpastes, toothpowders and liquid dentifrices, mouthwashes, troches, chewing gums, dental pastes, gingival massage creams, gargle tablets, dairy products and other foodstuffs. An oral composition according to this invention may further include additional well known ingredients depending on the type and form of a particular oral composition.

Optionally, the composition may further include one or more antibiotics that are toxic to or inhibit the growth of Gram negative anaerobic bacteria. Potentially any bacteriostatic or bactericidal antibiotic may be used in a composition of the invention. Preferably, suitable antibiotics include amoxicillin, doxycycline or metronidazole.

In certain preferred forms of the invention the oral composition may be substantially liquid in character, such as a mouthwash or rinse. In such a preparation the vehicle is typically a water-alcohol mixture desirably including a humectant as described below.

Generally, the weight ratio of water to alcohol is in the range of from about 1:1 to about 20:1. The total amount of water-alcohol mixture in this type of preparation is typically in the range of from about 70 to about 99.9% by weight of the preparation. The alcohol is typically ethanol or isopropanol. Ethanol is preferred.

The pH of such liquid and other preparations of the invention is generally in the range of from about 5 to about 9 and typically from about 5.0 to 7.0. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc).

In other desirable forms of this invention, the composition may be substantially solid or pasty in character, such as toothpowder, a dental tablet or a toothpaste (dental cream) or gel dentifrice. The vehicle of such solid or pasty oral preparations generally contains dentally acceptable polishing material.

In toothpaste, the liquid vehicle may comprise water and humectant typically in an amount ranging from about 10% to about 80% by weight of the preparation. Glycerine, propylene glycol, sorbitol and polypropylene glycol exemplify suitable humectants/carriers. Also advantageous are liquid mixtures of water, glycerine and sorbitol. In clear gels where the refractive index is an important consideration, about 2.5-30% w/w of water, 0 to about 70% w/w of glycerine and about 20-80% w/w of sorbitol are preferably employed.

Toothpaste, creams and gels typically contain a natural or synthetic thickener or gelling agent in proportions of about 0.1 to about 10, preferably about 0.5 to about 5% w/w. A suitable thickener is synthetic hectorite, a synthetic colloidal magnesium alkali metal silicate complex clay available for example as Laponite (e.g. CP, SP 2002, D) marketed by Laporte Industries Limited. Laponite D is, approximately by weight 58.00% SiO₂, 25.40% MgO, 3.05% Na₂O, 0.98% Li₂O, and some water and trace metals. Its true specific gravity is 2.53 and it has an apparent bulk density of 1.0 g/ml at 8% moisture.

Other suitable thickeners include Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose (e.g. available as Natrosol), sodium carboxymethyl cellulose, and colloidal silica such as finely ground Syloid (e.g. 244). Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive, oil, castor oil and petrolatum and esters such as amyl acetate, ethyl acetate and benzyl benzoate.

It will be understood that, as is conventional, the oral preparations will usually be sold or otherwise distributed in suitable labelled packages. Thus, a bottle of mouth rinse will have a label describing it, in substance, as a mouth rinse or mouthwash and having directions for its use; and a toothpaste, cream or gel will usually be in a collapsible tube, typically aluminium, lined lead or plastic, or other squeeze, pump or pressurized dispenser for metering out the contents, having a label describing it, in substance, as a toothpaste, gel or dental cream.

Organic surface-active agents may be used in the compositions of the present invention to achieve increased therapeutic or prophylactic action, assist in achieving thorough and complete dispersion of the active agent throughout the oral cavity, and render the instant compositions more cosmetically acceptable. The organic surface-active material is preferably anionic, non-ionic or ampholytic in nature and preferably does not interact with the active agent. It is preferred to employ as the surface-active agent a detersive material which imparts to the composition detersive and foaming properties. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkylsulfo-acetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarconite compounds in the oral compositions of the present invention is particularly advantageous since these materials exhibit a prolonged marked effect in the inhibition of acid formation in the oral cavity due to carbohydrates breakdown in addition to exerting some reduction in the solubility of tooth enamel in acid solutions. Examples of water-soluble non-ionic surfactants suitable for use are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly (ethylene oxide) with fatty acids, fatty alcohols, fatty amides, polyhydric alcohols (e.g. sorbitan monostearate) and polypropyleneoxide (e.g. Pluronic materials).

The surface active agent is typically present in amount of about 0.1-5% by weight. Various other materials may be incorporated in the oral preparations of this invention such as whitening agents, preservatives, silicones, chlorophyll compounds and/or ammoniated material such as urea, diammonium phosphate, and mixtures thereof. These adjuvants, where present, are incorporated in the preparations in amounts which do not substantially adversely affect the properties and characteristics desired.

Any suitable flavouring or sweetening material may also be employed. Examples of suitable flavouring constituents are flavouring oils, e.g. oil of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, xylitol, sodium cyclamate, perillartine, AMP (aspartyl phenyl alanine, methyl ester), saccharine, and the like. Suitably, flavour and sweetening agents may each or together comprise from about 0.1% to 5% more of the preparation.

The compound, peptide or peptidomimetic of composition of the invention can also be incorporated in lozenges, or in chewing gum or other products, e.g. by stirring into a warm gum base or coating the outer surface of a gum base, illustrative of which are jelutong, rubber latex, vinylite resins, etc., desirably with conventional plasticizers or softeners, sugar or other sweeteners or such as glucose, sorbitol and the like.

The invention provides a method for treating or alleviating the symptoms of periodontal disease in a subject, the method comprising administering to the subject a compound, peptide, peptidomimetic or composition of the invention and a protein for inducing an immune response to P. gingivalis. The protein for inducing an immune response to P. gingivalis includes those proteins described in PCT/AU2009/001112 (WO/2010/022463) which is herein incorporated by reference.

In a further aspect, the present invention provides a kit of parts including (a) a compound, peptide, peptidomimetic or composition and (b) a pharmaceutically acceptable carrier. Desirably, the kit further includes instructions for their use for the treatment or prevention of periodontal disease in a patent in need of such treatment.

Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

The aqueous suspensions may also contain one or more preservatives, for example benzoates, such as ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

To describe the invention in more detail, the following examples are described to illustrate some aspects and embodiments of the invention.

### EXAMPLE 1

### Preparation of Kgp Propeptide and Inhibition of Kgp Activity

### Bacterial Strains and Growth Conditions

Glycerol or freeze-dried cultures of Porphyromonas gingivalis W50 and the Kgp_{cat}ΔABM1 mutant ECR368 were grown anaerobically at 37° C. on Horse Blood Agar (HBA; Oxoid). P. gingivalis was maintained by passage and only passage 3-7 were used to inoculate 20 mL and 200 mL Brain Heart Infusion broth (37 g/L), supplemented with hemin (5 mg/L) and cysteine (0.5 g/L) and erythromycin supplementation (10 μg/mL) for ECR368 (BHI). Growth was determined by measurement of culture optical density (OD) at a wavelength of 650 nm. Gram stains of the cultures were carried out to check for any contamination. The P. gingivalis cells were harvested during exponential growth phase by centrifugation (8000 g, 20 min, 4° C.) and washed once with TC150 buffer (50 mM Tris-HCl, 150 mM NaCl, 5 mM CaCl₂, pH 8.0) containing 0.5 g/L cysteine. The washed cells were resuspended in 2 mL of TC150 buffer (with 0.5 g/L cysteine), and kept at 4° C. to be used immediately in the proteolytic assays.

P. gingivalis W50 was grown in a minimal medium for at least 6 passages and stored in −80° C. for subsequent growth experiments. The minimal medium was prepared as follows: basal buffer (10 mM NaH₂PO₄, 10 mM KCl, and 10 mM MgCl₂) was supplemented with haemoglobin (50 nM) and BSA (3% A-7906; Sigma-Aldrich Co.), pH 7.4, and filter sterilized (0.1 μm membrane filter Filtropur BT50, Sarstedt). The cells (10⁸ in 200 μL) were inoculated into each well of the 96-well microtitre plate (Greiner Bio-One 96-Well Cell Culture Plates) with 100 mg/L of Kgp propeptide (Kgp-PP), RgpB propeptide (RgpB-PP) or Kgp-PP plus RgpB-PP. The plate was incubated overnight at 37° C. in the anaerobic chamber, sealed with a plateseal microtitre plate sealer (Perkin Elmer Life Sciences, Rowville, VIC, Australia). The absorbance was monitored at 620 nm for 50 h at 37° C., using a microplate reader (Multiskan Ascent microplate reader—Thermo Electron Corporation). The P. gingivalis W50 isogenic triple mutant lacking RgpA, RgpB, and Kgp was used as a negative control of growth in the minimal medium. The growth in presence of propeptide was compared against the growth of P. gingivalis in the minimal medium.

### Purification of Lys-Gingipain (Kgp)

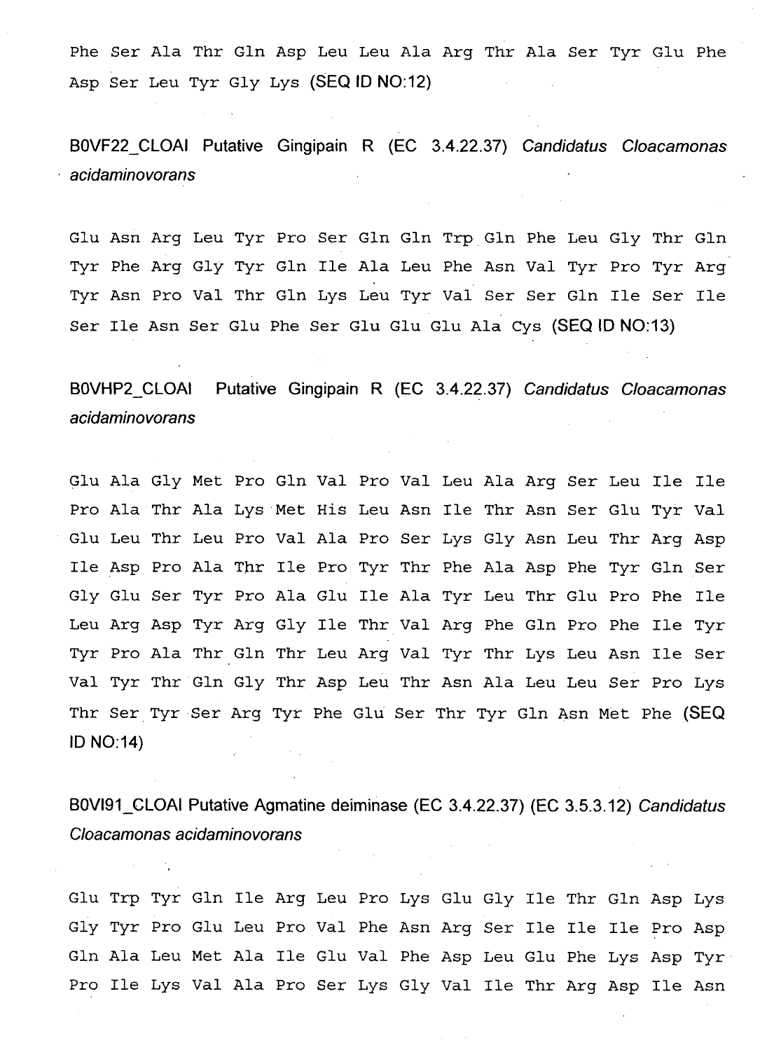

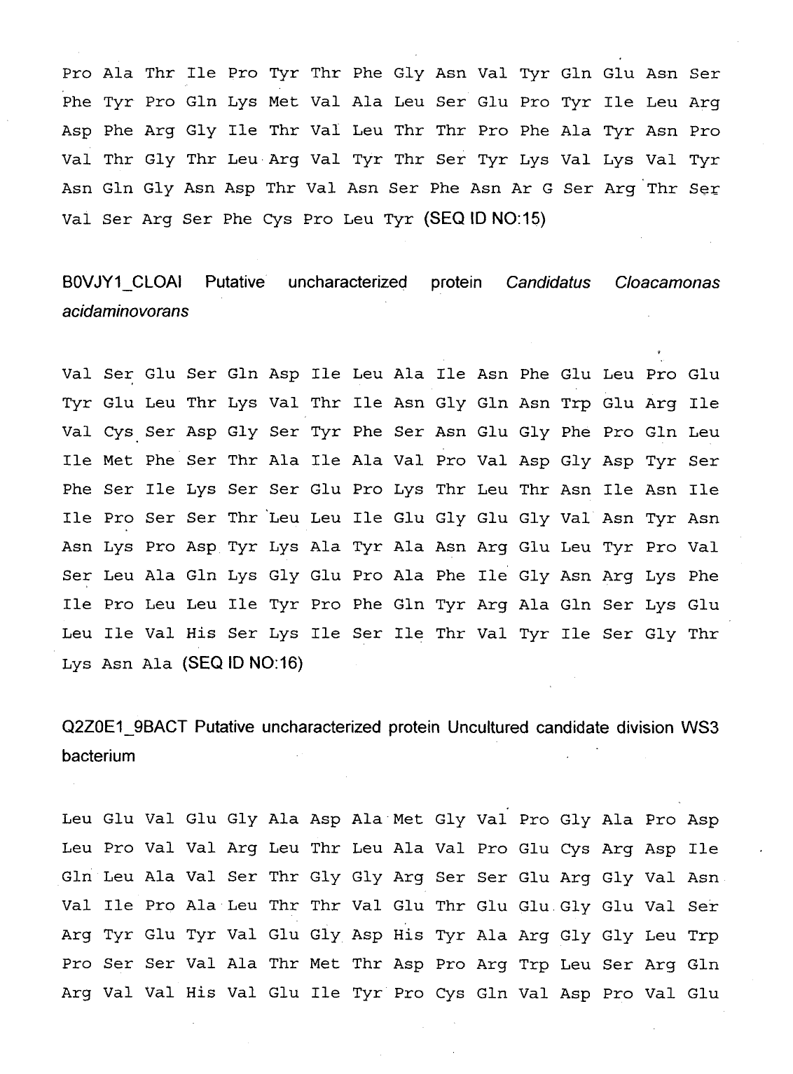

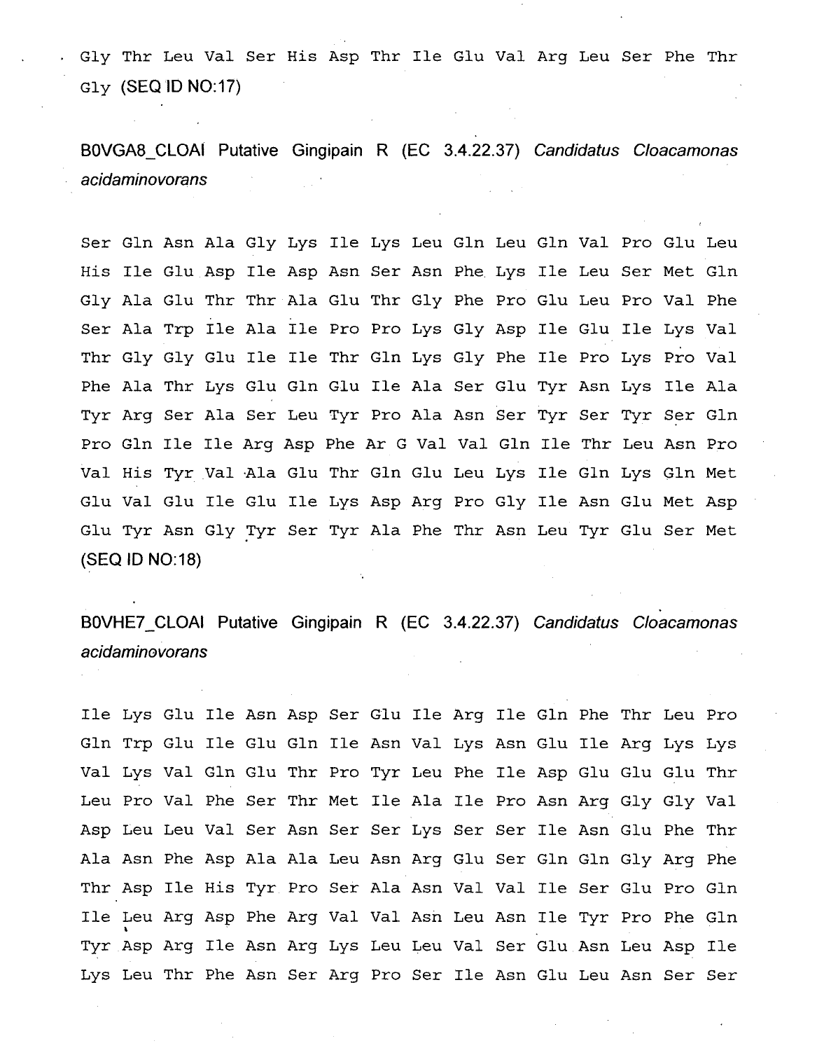

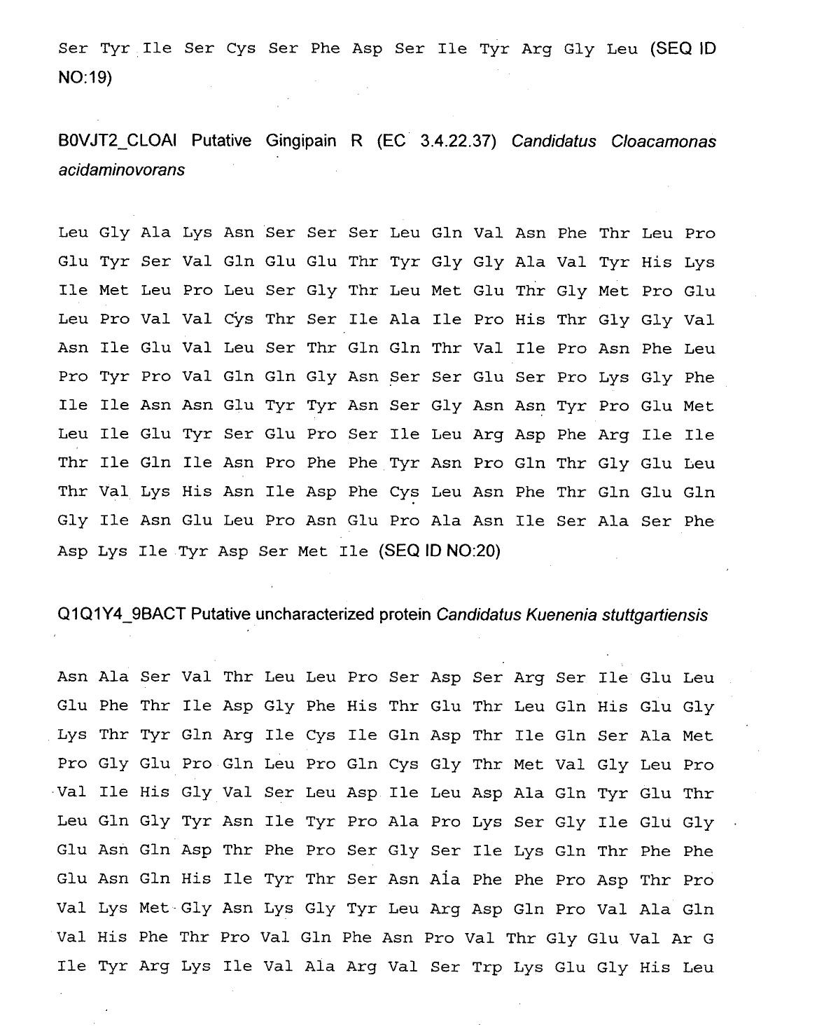

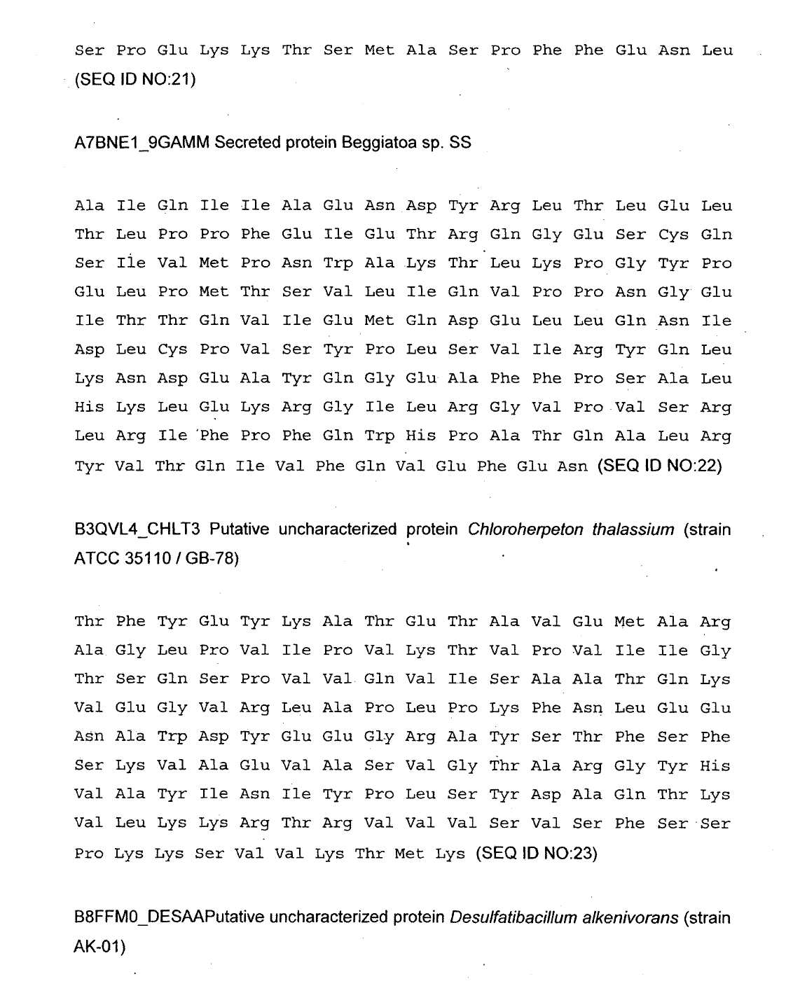

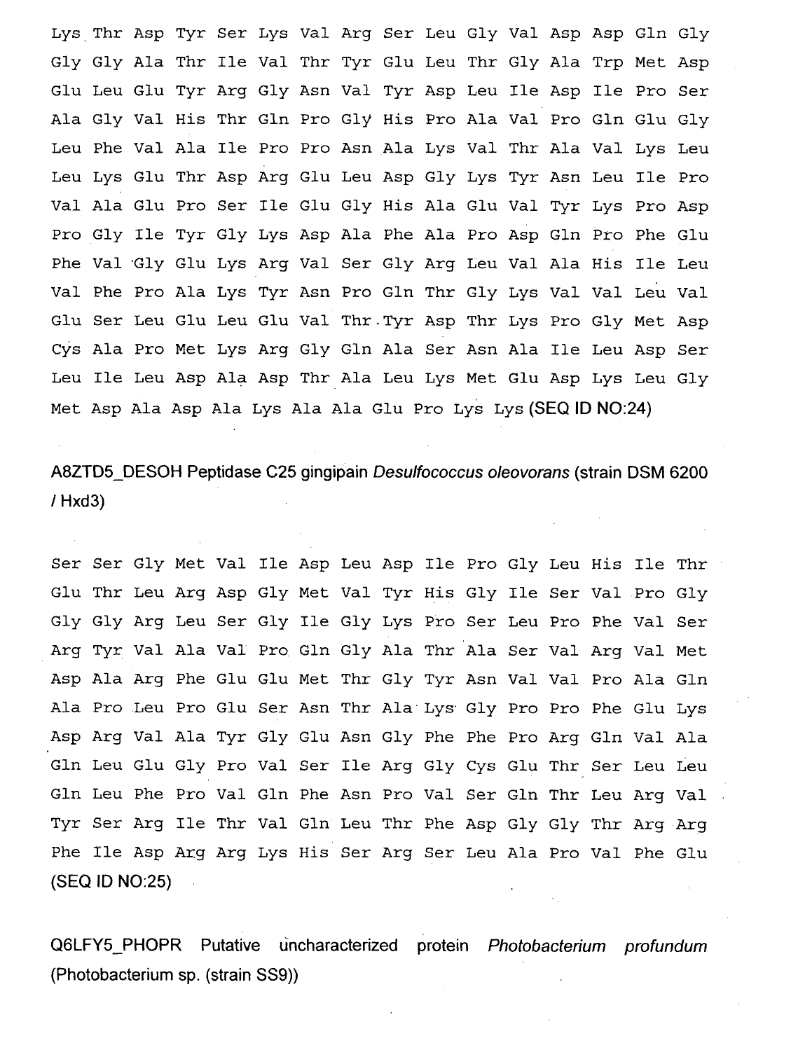

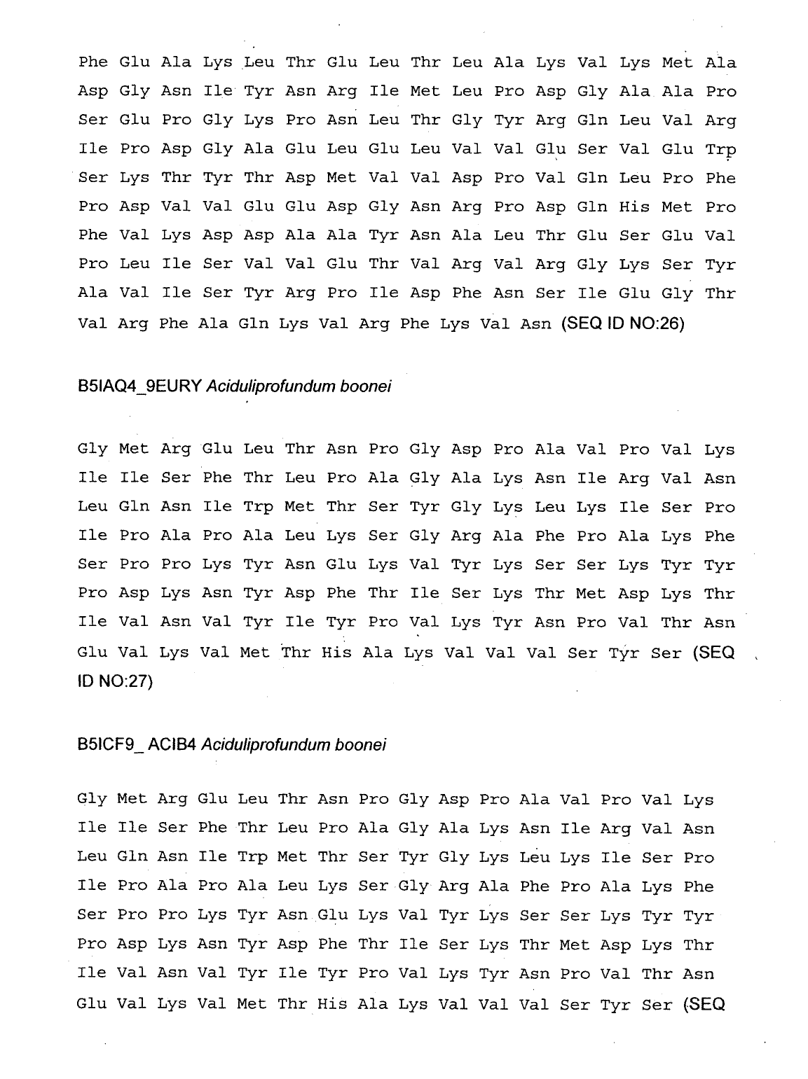

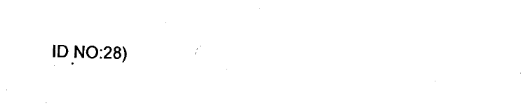

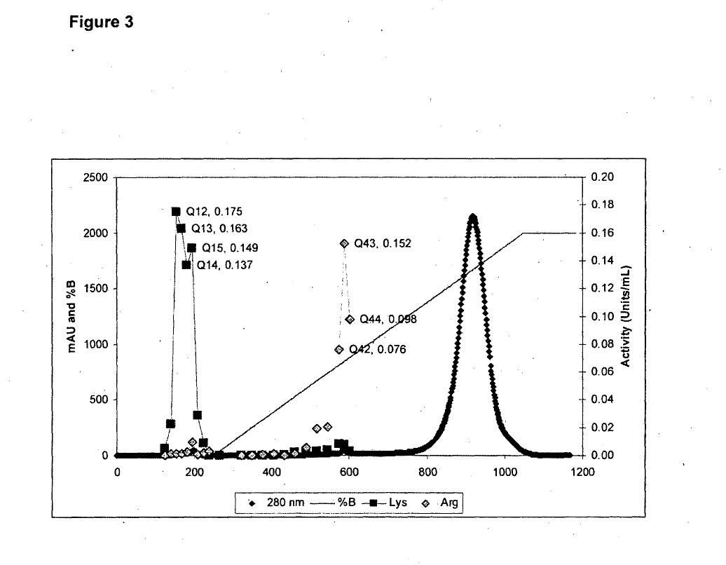

For harvesting and purification of the mature Kgp_{cat}ΔABM1, 4 mL of the Kgp_{cat}ΔABM1 mutant ECR368 starter culture was used to inoculate 200 mL BHI broth that was then incubated over three days at 37° C. The P. gingivalis cells were first removed by centrifugation at 8,000 g for 30 min at 4° C. after which the supernatant was collected and ultracentrifuged at 100,000 g for 1 h at −10° C. to remove vesicles. The pellets were discarded and the supernatant was collected and stored on an ice/salt mixture. Chilled acetone was slowly added to the chilled supernatant in a 3:2 ratio v/v and the proteins precipitated by centrifugation (8,000 g for 30 min, −10° C.). The supernatant was carefully discarded and the precipitate washed in TC50 buffer (50 mM Tris-HCl, 50 mM NaCl, 5 mM CaCl₂, pH 7.4). After centrifugation (8,000 g for 30 min, −10° C.), the precipitate was resuspended in TC50 buffer and filtered through a 0.22 μM filter. This extract was applied to a desalting column (Sephadex G25, XK26/40) attached to an AKTA-Basic FPLC system, and eluted with TC50 buffer at a flow rate of 5 mL/min. The eluate was monitored at 280 and 254 nm. The void volume was collected and concentrated to <10 mL by ultrafiltration using 10,000 MW cut-off membranes (Vivaspins). The concentrated sample was applied to an anion exchange column (Q-sepharose), to separate the fractions with Lys-activity from those with Arg-activity (FIG. 3). The pooled concentrated fractions with Lys-activity were then applied to a cation exchange column S-sepharose. The eluted fractions with Lys-activity were then size-fractionated using gel filtration column (Superdex G75, XK16/100) to separate Kgp proteases from the other proteins. The column was eluted with TC50 buffer at a flow rate of 1 mL/min. The eluate was monitored at 280, 254 and 215 nm, collected and stored at −70° C.

### Expression and Purification of Recombinant Kgp-Propeptide

The genomic DNA encoding the propeptide of Kgp (amino acids 20-228) was amplified by polymerase chain reaction (PCR) using the genomic DNA of Kgp as a template. Primers 5′ ACG CAG CAT ATG CAA AGC GCC AAG ATT AAG CTT GAT 3′ and 5′ ACG CAG CTC GAG TCA TCT ATT GAA GAG CTG TTT ATA AGC 3′ were used for PCR These primers contained the Nde1 and XhoI restriction sites. An additional stop codon site was designed at the antisense position. The size of the DNA was checked by SDS-PAGE and the PCR product was cloned into PGEM-T easy vector (Promega) using TA cloning kit (Invitrogen). The PCR insert was removed after cleavage with enzymes Nde1 and XhoI, purified by gel extraction then inserted into the PET-28b expression vector (Novagen). The insert was sequenced to verify correct amplification and ligation.

For expression in Escherichia coil BL-21 (DE3) (Novagen), the PET-28b vector was transformed into the BL-21 (DE3) cells. Expression was induced by addition of 1 mM Isopropyl β-D-1-thiogalactopyranoside (IPTG). After 4 h of induced expression, the cells were harvested by centrifugation at 8,000 g for 20 min. The cells, containing the recombinant propeptide in inclusion bodies, were suspended in lysis buffer (50 mM Na₂HPO₄, 300 mM NaCl, 10 mM imidazole, pH 8.0) and disrupted by sonication (15 min) and stirring (30 min, 4° C.). The lysate was centrifuged and the resulting supernatant purified using Ni affinity chromatography to obtain purified recombinant propeptide.

A 50% Ni-NTA (Qiagen) slurry (4 mL) was added to the supernatant, which was then stirred for 15 min at 4° C. The mixture was loaded on an open column with a bed volume of 20 mL and the flow through was removed. The resin was washed twice with 10 mL purification buffer (50 mM potassium phosphate at pH 8.0, 150 mM NaCl, 20 mM imidazole). Then purification buffer (2 mL) containing 25 NIH units of thrombin (Sigma) was added to the slurry and allowed to incubate for 2 h at room temperature to enable thrombin to cleave the propeptide from its His-tag and release it from the nickel affinity resin. The released propeptides with the thrombin protease were collected with 15 mL purification buffer. The solution was loaded onto another column containing 1 mL of Benzamidine Sepharose resin (Pharmacia) and allowed to react for 15 min at room temperature, to enable the thrombin protease to bind to the Benzamidine Sepharose resin. The flow through fraction was collected. The Benzamidine Sepharose resin was washed twice with 2.5 mL of wash buffer (5 mM potassium phosphate at pH 7.0, 50 mM NaCl), and the washes were collected. The flow through fraction was combined with the two wash fractions, resulting in a 20 mL solution that was lyophilised. The redissolved extract was applied to a gel filtration column (Superdex G75, XK16/100) attached to an AKTA-Basic FPLC system and eluted with 50 mM NH₄HCO₃ at a flow rate of 1 mL/min.

The eluate was monitored at 280 and 215 nm. The eluate was collected, lyophilised and stored at −70° C.

### Spectrophotometric Determination of Protein Concentration

The molar extinction coefficient (ε) (M⁻¹cm⁻¹) at 280 nm and the molecular weights of the proteins were determined using the “ProtParam” program on the ExPASy server (Gasteiger et al., 2005). The ε of Kgp_{cat}ΔABM1 was 105,340 M⁻¹cm⁻¹ while the ε of the rKgp propeptide was 11,920 M⁻¹cm⁻¹. The concentrations of the Kgp_{cat}ΔABM1 enriched fraction and the rKgp propeptide were determined using spectrophotometric means (Grimsley and Pace, 2003). The absorbance of each sample was measured by scanning the absorbance from 200 nm to 300 nm using the Varian Cary 50 Dual Beam spectrophotometer (Australia). The absorbance at 280 nm, which is absorbed by Trp, Tyr, and Cys residues, was used to calculate the protein concentration using Beer-Lamberts Law (A₂₈₀ₙₘ=εbC). The Kgp_{cat}ΔABM1 enriched fractions were subsequently diluted for the protease inhibition assays.

### MALDI TOF/TOF MS

Peptide samples were co-crystallized (1:1 vol/vol) on a MTP 384 target ground steel plate with saturated 2,5-dihydroxybenzoic acid (DHB) matrix in standard buffer (50% acetonitrile, 0.1% TFA). The samples were analysed on an Ultraflex MALDI TOF/TOF Mass Spectrometer (Bruker, Bremen, Germany). Analysis was performed using Bruker Daltonics flexAnalysis 2.4 and Bruker Daltonics BioTools 3.0 software with fragmentation spectra matched to a casein database installed on a local MASCOT server.

### Electrospray MS

Fractions collected from the RP-HPLC were analysed using an Esquire-LC MS/MS system (Bruker Daltonics) operating in the electrospray mass spectrometry mode. Sample injection was conducted at 340 μL/h, with nitrogen flow of 5 L/min and drying gas temperature of 300° C.

### Protease Inhibition Assays

Lys-specific proteolytic activity was determined using synthetic chromogenic substrate N-(p-Tosyl)-Gly-Pro-Lys 4-nitroanilide acetate salt (GPK-NA) (Sigma Aldrich). The Lys-specific reaction buffer contained 2 mM GPK-NA dissolved in 30% v/v isopropanol, 0.93 mM cysteine, 400 mM Tris-HCl pH 8.0, and 100 mM NaCl. Protease assays were conducted in sterile 96-well microtitre plates (Corning Incorporated, NY) with all fractions and controls assayed in triplicate. The rKgp propeptides were added to the wells in a final concentration of 20.0 mg/L (0.85 μM) and 40.0 mg/L (1.71 μM) with 10 μL of 10 mM cysteine pH 8.0 and a final concentration of 1.16 mg/L (0.02 μM) Kgp_{cat}ΔABM1 enriched fraction, topped-up to a volume of 100 μL with TC150 buffer (50 mM Tris-HCl, 150 mM NaCl, 5 mM CaCl₂, pH 8.0). Samples were incubated at 37° C. for 15 min before the addition of 100 μL of chromogenic substrate (2 mM) (total volume 200 μL). Protease activity was determined by measuring the absorbance at 405 nm with 10 s intervals for ˜20 min at 37° C., pH 8.0 using a PerkinElmer 1420 Multilabel Counter VICTOR3™. Kgp_{cat}ΔABM1 enriched fraction proteolytic activity was determined as Units/mg.

Bacterial protease inhibitory activity was also determined using DQ™ Green bovine serum albumin (BSA) (Molecular Probes, USA) (Grenier et al., 2001; Yoshioka et al., 2003). The protein is labelled with a strong self-quenched amine dye which when cleaved emits maximally at 535 nm following excitation at 485 nm. The assay mixture contained Kgp_{cat}ΔABM1 enriched fraction (1.16 mg/L, 0.02 μM), the rKgp propeptides (40.0 mg/L), 1 mM cysteine, and DQ BSA (10 μL; 0.1 g/L), made up to a final volume of 200 μL with TC150 buffer. Nα-p-tosyl-l-lysine chloromethylketone TLCK (1 mM) treated Kgp_{cat}ΔABM1 proteases were used as a control. TLCK is a strong cysteine protease inhibitor known to inhibit both Rgp and Kgp activity (Fletcher et al., 1994; Pike et al., 1994). Leupeptin, an Rgp inhibitor was added to the assay to inhibit any Arg-gingipain activity that may be present (Kitano et al., 2001). The assay mixtures were incubated in the dark for 2 h at 37° C. prior to measuring the fluorescence which indicates the degree of albumin degradation, using a fluorometer (PerkinElmer 1420 Multilabel Counter VICTOR3™). The fluorescence value obtained with the negative control (TLCK-treated) was subtracted from all values. All assays were performed in triplicate with 2-3 biological replicates unless stated otherwise, and the mean±standard deviation was calculated.

Samples from each well were analysed for propeptide and protease hydrolysis using reversed phase-high performance liquid chromatography (RP-HPLC) and SDS-PAGE. 200 μL of each sample was analysed on an analytical Zorbax 300 SB-C₁₈ reversed phase column (4.6 mm×250 mm) connected to an Agilent Preparative 1100 HPLC instrument (Agilent Technologies) using a flow rate of 1 mL/min and a gradient of 0-100% solvent B (90% acetonitrile-0.1% (v/v) TFA in deionised water) in 30 min. For SDS-PAGE analysis, each assay sample (200 μL) was centrifuged at 14,500 rpm for 5 min, then 50 μL of the supernatant was denatured with 5% (v/v) 1 M DTT and 25% (v/v) 4× reducing sample buffer, heated for 10 min at 70° C. and briefly microcentrifuged before being loaded onto a precast 8-12% gradient Bis-Tris gel. SeeBlue® Pre-Stained standard was used as a molecular marker and a potential difference of 150 V and MES buffer were used to run the gel. The gel was stained with Coomassie® Brilliant Blue (G250) overnight and destained in deionised water. The gel was scanned using an Epson Smart Panel scanner connected to a Proteineer SP system (Bruker Daltonics).

### Statistical Analysis

Protease activity data were subjected to a single factor analysis of variance (ANOVA). When the ANOVA indicated statistical significant difference (p<0.05) between the means of tested inhibitors, a modified Tukey test was performed on the data to identify which inhibitors were significantly different (Zar, 1984; Fowler and Cohen, 1997).

### Molecular Modelling

The program Fugue (Shi et al., 2001) was used to identify possible structure motifs for the three gingipain propeptides against a curated protein database HOMSTRAD (Mizuguchi et al., 1998). The program PSI-BLAST was run concurrently to identify any other putative orthologs and paralogs.

### Purification of Lys-Gingipain (Kgp)

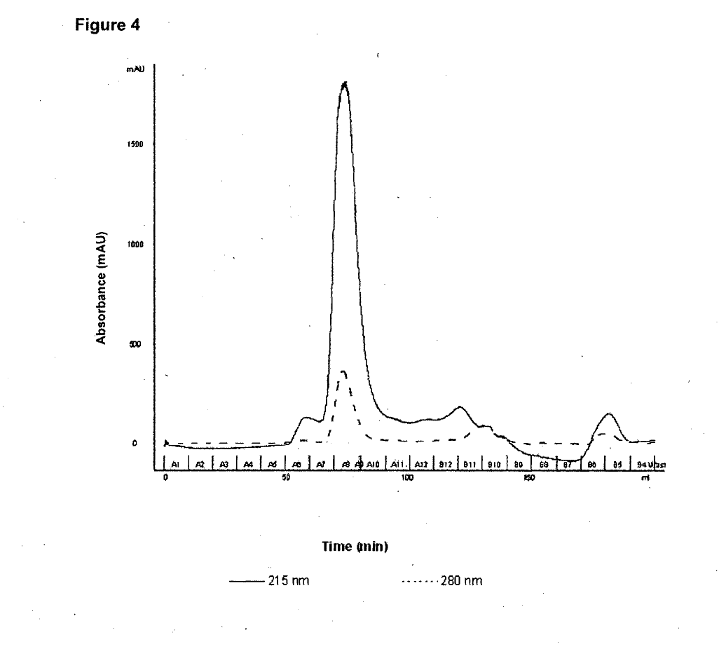

P. gingivalis ECR368 was grown anaerobically for 3 days and the culture supernatant harvested for Kgp_{cat}ΔABM1 by acetone precipitation and centrifugation. The acetone precipitated proteins were loaded onto a desalting column (Sephadex G25) and eluted by 50 mM sodium acetate pH 5.3 buffer. The first peak was collected and concentrated using a 10,000 MW cut-off membrane. This extract was subjected to anion exchange and cation exchange chromatography and finally size-exclusion chromatography (FIG. 4) to separate the Kgp_{cat}ΔABM1 from other proteins in the supernatant. Samples from each purification step were analysed using SDS-PAGE gels for enzyme purity (FIG. 5). The purity of the Kgp_{cat}ΔABM1 increased with each subsequent purification step resulting in a Kgp_{cat}ΔABM1 enriched fraction (lane 5; FIG. 5).

### Expression and Purification of Recombinant Kgp Propeptide (rKgp)

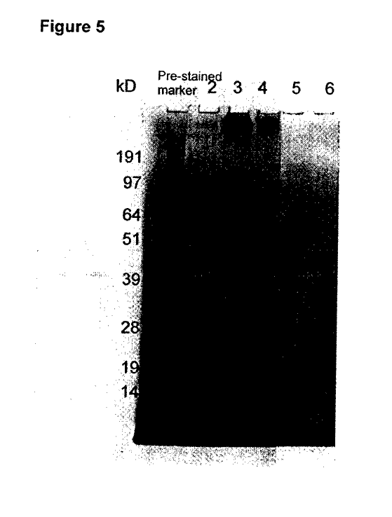

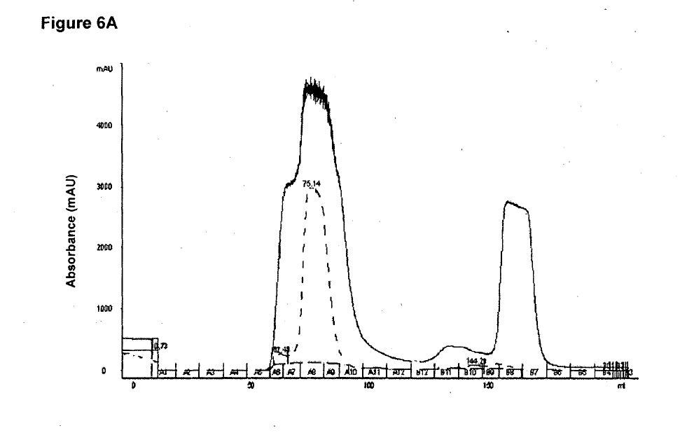

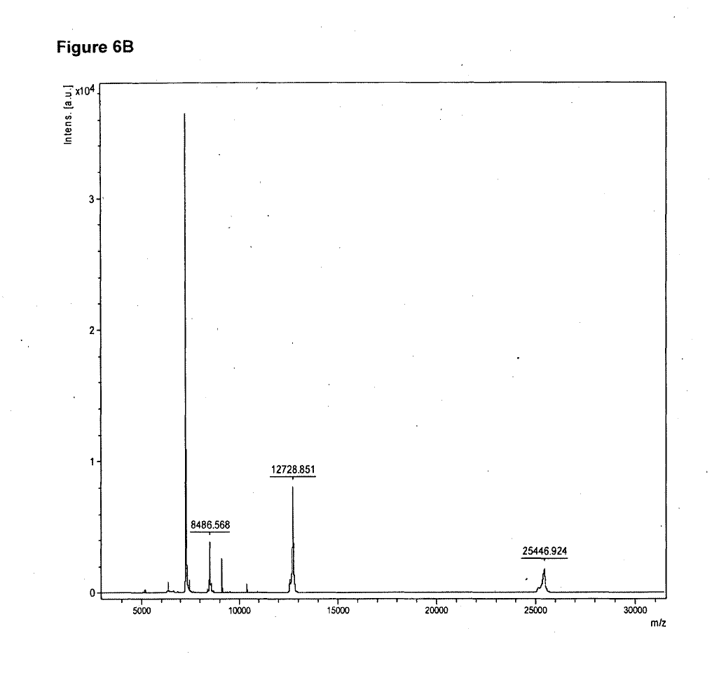

The rKgp propeptide was designed using a His-Tag sequence followed by a thrombin cleavage site, N-terminal to the propeptide. The rKgp propeptide was expressed in E. coli and extracted using Ni affinity chromatography of the cell lysate. To remove the His-tag, the E. coli cell lysate bound to the Ni-column was treated with thrombin which cleaved the propeptide leaving the His-tag attached to the Ni-column. The released propeptides were collected and applied to an open column with Benzamidine Sepharose to remove the thrombin protease followed by a gel filtration column to purify the rKgp propeptide (FIG. 6A). The identity of the rKgp propeptide was determined using MALDI-TOF MS analysis (FIG. 6B).

### Spectrophotometric Determination of Protein Concentration

The concentrations of the Kgp_{cat}ΔABM1 enriched fraction (MW 50, 114 Da, 454 aa) and the rKgp propeptides were determined using spectrophotometric means (Grimsley and Pace, 2003). The absorbance at 280 nm (A280 nm) of the Kgp_{cat}ΔABM1 enriched fraction was 0.033 and the extinction coefficient was 105,340 M⁻¹cm⁻¹; therefore the concentration of the Kgp_{cat}ΔABM1 enriched fraction was 0.0157 g/L. Several batches of Kgp_{cat}ΔABM1 enriched fractions were analysed for its protein concentration by A280 nm. However, the final concentration of Kgp_{cat}ΔABM1 enriched fraction in each assay was set as 1.16 mg/L (0.02 μM).

The concentration of the rKgp propeptides was determined in the same manner. The A280 nm of the rKgp propeptide (MW 23,403, 213 aa) was 0.1169, and has an extinction coefficient of 11,920 M⁻¹cm⁻¹ and therefore a concentration of 0.23 g/L. The final concentration of rKgp propeptide in the assays was 20.0 (0.85 μM) and 40.0 mg/L (1.71 μM).

### Protease Inhibition Assay

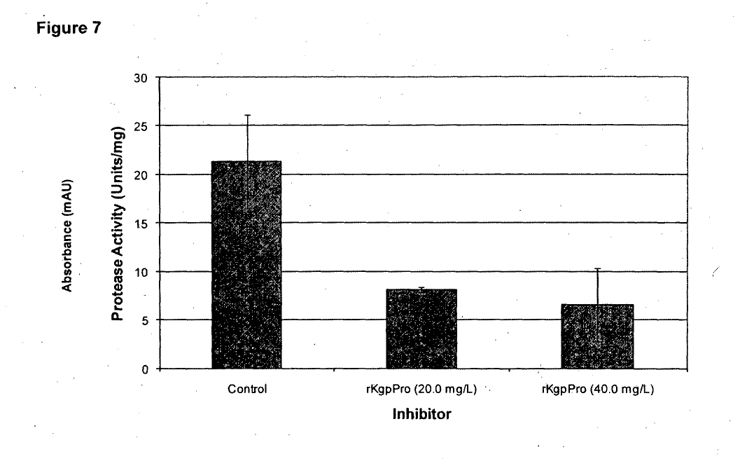

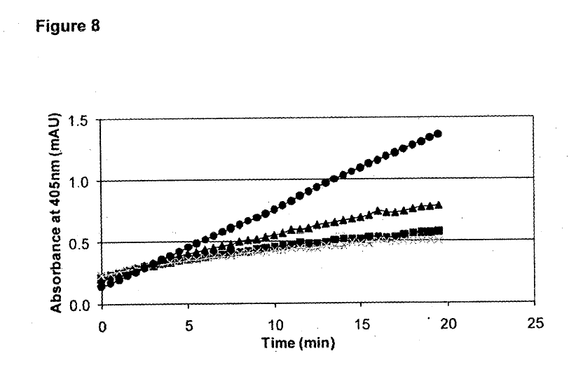

The inhibition of Kgp_{cat}ΔABM1 by the rKgp propeptides was determined using chromogenic and fluorescent substrates. In the chromogenic substrate assay, the final concentrations of rKgp propeptides were 20.0 mg/L (0.85 μM) and 40.0 mg/L (1.71 μM) and the concentration of Kgp_{cat}ΔABM1 enriched fraction was 1.16 mg/L (0.02 μM). The control used was TLCK at a concentration of 1 mM. The rKgp propeptide exhibited ˜75% inhibition of Kgp_{cat}ΔABM1 activity at a concentration of 40.0 mg/L (1.71 μM) while 20.0 mg/L (0.85 μM) rKgp propeptide inhibited ˜60% Kgp_{cat}ΔABM1 activity (FIG. 7). The rate of substrate hydrolysis was linear throughout the assay (FIG. 8).

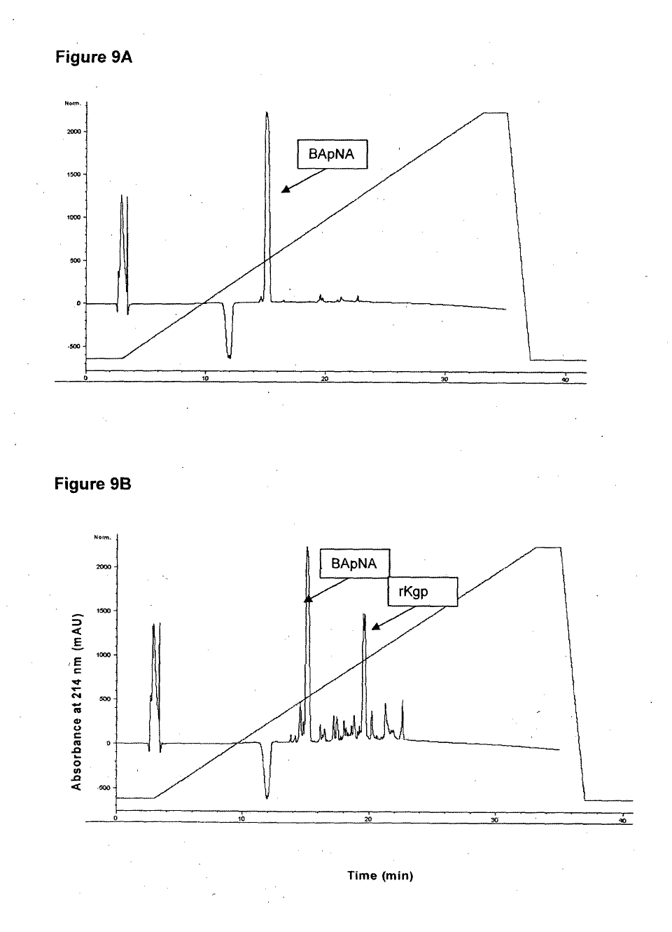

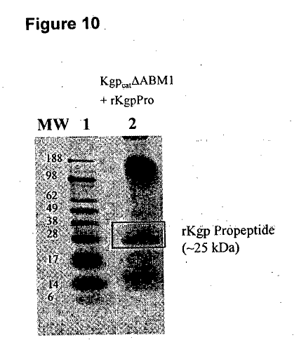

Samples from these assays were collected and analysed using RP-HPLC to determine potential hydrolysis of the rKgp propeptide or the Kgp_{cat}ΔABM1. The HPLC profiles indicated that the rKgp propeptide was still intact (FIG. 9). The samples (200 μL) were centrifuged and 50 μL of the supernatant was treated with DTT and sample buffer and analysed by SDS-PAGE. The SDS-PAGE analysis demonstrated that the propeptide was still present at the expected molecular weight (FIG. 10).

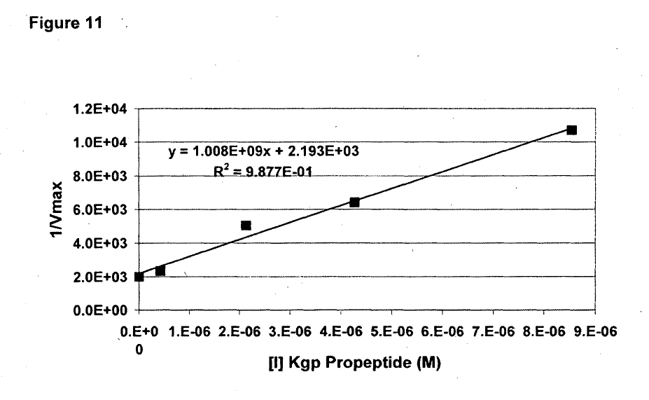

The inhibition kinetics of the rKgp propeptide against Kgp_{cat}ΔABM1 determined using the chromogenic substrate GPKNa revealed non-competitive inhibition. The Ki′ for Kgp propeptide was calculated to be 2.01 μM (FIG. 11).

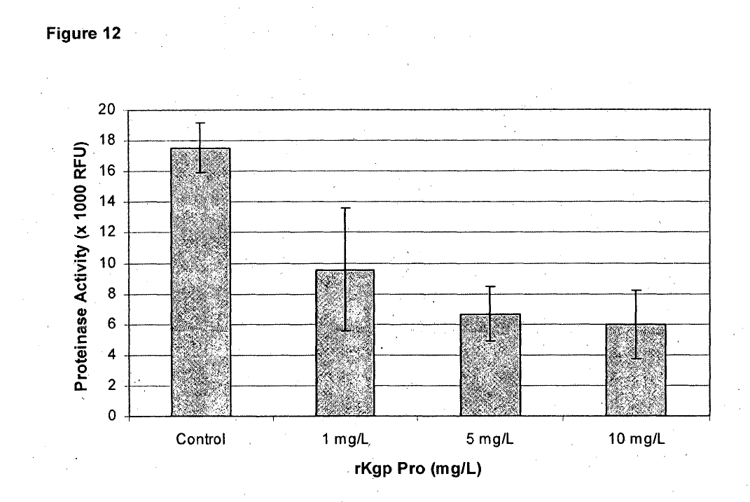

The fluorescent BSA substrate assays were performed within a 2 h incubation period. The rKgp propeptide exhibited ˜66% inhibition of Kgp_{cat}ΔABM1 enriched fraction activity at a concentration of 10.0 mg/L (0.45 μM) (FIG. 12). However, this assay measures total protease activity, so the rKgp propeptide inhibition of Kgp_{cat}ΔABM1 is underestimated due to the residual presence of RgpA that will cleave BSA.

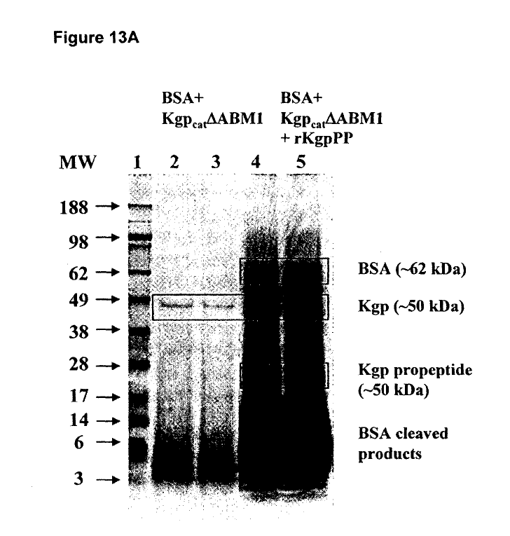

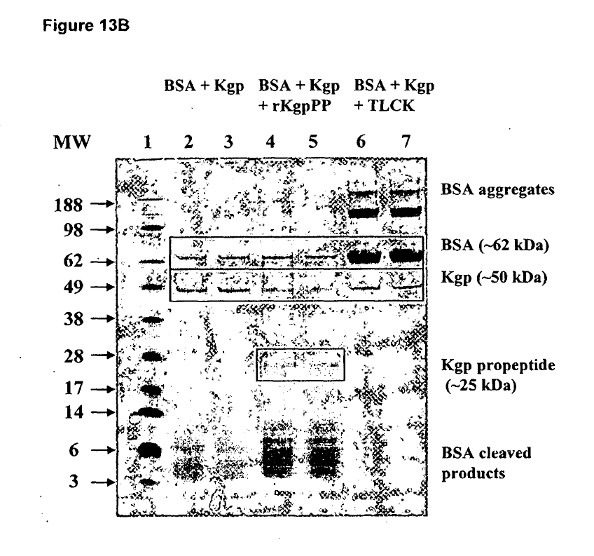

Samples from the assays were collected and analysed by SDS-PAGE (FIG. 13A). The control contains ˜0.03 μg Kgp_{cat}ΔABM1 and ˜1 μg BSA. A pellet was observed in the centrifuged samples containing rKgp propeptide and Kgp_{cat}ΔABM1 enriched fraction while no pellets were observed in the centrifuged samples just containing Kgp_{cat}ΔABM1 enriched fraction (control). These pellets were resuspended in supernatant and applied to SDS gels. The SDS gels indicate that the Kgp_{cat}ΔABM1 (MW ˜50,000) and the rKgp propeptides (MW ˜25,000) were still intact after the 2 h incubation period (FIGS. 13A & 13B). The presence of BSA (MW 62,000) and its cleaved products were also observed on the gel. The Kgp_{cat}ΔABM1 cleaved all BSA into small peptides that were difficult to detect as those cleavage products most likely ran off the end of the gel (FIG. 13A, lanes 2 and 3) while intact BSA was still present when rKgp propeptide was added to Kgp_{cat}ΔABM1 (lanes 4 and 4) and the cleaved peptides were still relatively large, between about 14 and 3kDa, indicating an inhibition of Kgp protease activity by the propeptide. The TLCK controls indicate that the proteases were inhibited and no BSA degradation was observed (FIG. 13B).

### EXAMPLE 2

### Preparation of RgpB Propeptide and Inhibition of Rgp Activity

### Growth Conditions for P gingivalis HG66

Glycerol cultures of P. gingivalis strain HG66 were grown anaerobically at 37° C. in an anaerobe chamber, with an atmosphere of 10% CO₂, 5% H₂, 85% N₂, on horse blood agar (HBA; Oxoid). P. gingivalis cultures were maintained by passages weekly until 7-10 passages were completed, after which a fresh culture was recovered from glycerol stocks. To grow P. gingivalis in broth culture, a starter culture was prepared by inoculation of several colonies (selected from a 5-7 day old plate) into 20 mL BHI broth (Brain Heart Infusion broth (37 g/L), supplemented with haemin (5 mg/L), cysteine (0.5 g/L), vitamin K₃ (menadione) (5 mg/L) before being incubated overnight at 37° C. Culture purity was routinely assessed by Gram stain and observation of colony morphology on HBA plates.

### Purification of Arg-Gingipain (RgpB)

For harvesting and purification of the mature RgpB, 40 mL of starter culture was used to inoculate 2 L BHI broth which was then incubated over three-four days at 37° C. The P. gingivalis cells were removed by centrifugation at 17,700 g for 1 h at 4° C., after which the supernatant was collected and the pH adjusted to pH 5.3 with 50 mM Sodium Acetate then filtered through 0.8/0.2 μM filters to remove vesicles (contained in the pellets). The supernatant was poured off, collected and stored on an ice/salt mixture; chilled acetone was slowly added to the chilled supernatant in a 3:2 ratio v/v and the precipitated proteins collected by centrifugation (8,000 g for 30 min, −10° C.). The supernatant was carefully discarded and the precipitate was redissolved in NaOAc pH 5.5 buffer. After centrifugation (8,000 g for 30 min, −10° C.), the supernatant was filtered through a 0.22 μM filter. This extract was applied to a gel filtration column (Superdex G75, XK16/100) attached to an AKTA-Basic FPLC system, to separate the gingipains from the other proteins. The column was eluted with NaOAc pH 5.5 buffer at a flow rate of 0.5 mL/min, with the eluate being monitored at 280, 254 and 215 nm and the resulting fractions collected and stored at −70° C.

### Expression and Purification of Recombinant RgpB-Propeptide

The genomic DNA encoding the propeptide of RgpB was amplified by polymerase chain reaction (PCR) using the genomic DNA of RgpB as a template. Primers 5′ ACG CAG CAT ATG CAA AGC GCC AAG ATT AAG CTT GAT 3′ and 5′ ACG CAG CTC GAG TCA TCT ATT GAA GAG CTG TTT ATA AGC 3′ were used for PCR. These primers contained the Nde1 and XhoI restriction sites. An additional stop codon site was designed at the antisense position. The size of the DNA was checked by SDS-PAGE and the PCR product was cloned into pGEM-T Easy vector (Promega) using TA cloning kit (Invitrogen). The PCR insert was removed after cleavage with enzymes Nde1 and XhoI, purified by gel extraction then inserted into the PET-28b expression vector (Novagen). The insert was sequenced to verify correct amplification and ligation.

For expression in E. coli BL-21 (DE3) (Novagen), the PET-28b vector was transformed into the BL-21 (DE3) cells. Expression was induced by addition of 1 mM Isopropyl β-D-1-thiogalactopyranoside (IPTG). After 20 h, 15° C., of induced expression, the cells were harvested by centrifugation at 8,000 g for 20 min. The cells were suspended in lysis buffer (50 mM Na₂HPO₄, 300 mM NaCl, 10 mM imidazole, pH 8.0) and then disrupted by sonication (20 min) and stirring (30 min, 4° C.). The lysate was centrifuged and the resulting supernatant purified using Ni affinity chromatography to obtain purified recombinant propeptide.

A 50% Ni-NTA (Qiagen) slurry (4 mL) was added to the supernatant, stirred for 15 min at 4° C. and loaded on an open column with a bed volume of 20 mL, the flow through was removed. The resin was washed twice with 10 mL purification buffer (50 mM potassium phosphate at pH 8.0, 150 mM NaCl, and 20 mM imidazole). Purification buffer (2 mL) containing 25 NIH units of thrombin (Sigma) was added to the slurry and incubated for 2 h at room temperature. The released propeptides and thrombin protease were washed from the column using 15 mL purification buffer, and this solution was loaded onto another column containing 1 mL of Benzamidine Sepharose resin (Pharmacia). The solution was left to react for 15 min at room temperature to enable the thrombin protease to bind to the Benzamidine Sepharose resin. Once the flow through fraction was collected, the Benzamidine Sepharose resin was then washed twice with 2.5 mL of wash buffer (5 mM potassium phosphate at pH 7.0, 50 mM NaCl), with each of the washes collected too. The flow through fraction was then combined with the two wash fractions, resulting in a 20 mL solution that was lyophilised. The redissolved extract was applied to a gel filtration column (Superdex G75, XK16/100) attached to an AKTA-Basic FPLC system and eluted with 50 mM NH₄HCO₃ at a flow rate of 1 mL/min. The eluate was monitored at 280 and 215 nm. The eluate was collected, lyophilised and stored at −70° C.

### Protease Inhibition Assay

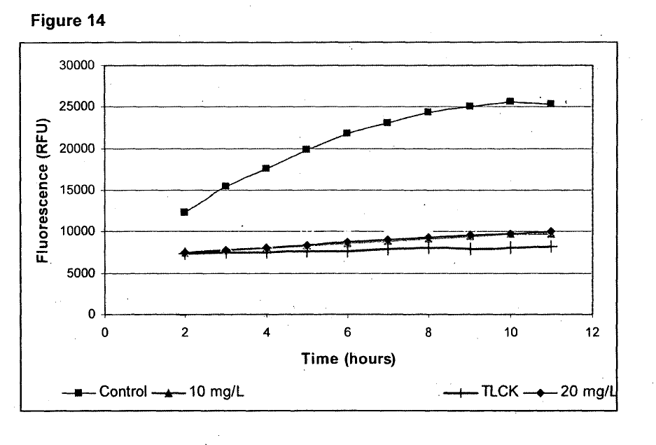

The proteolytic activity of the RgpB was determined in an assay using a fluorescent DQ-BSA substrate. Fluorescence was measured over 11 hours at 37° C. with a reading taken every hour. Addition of 10 mg/L (0.44 μM) or 20 mg/L (0.88 μM) RgpB propeptide resulted in near total inhibition of RgpB proteolytic activity over the entire length of the assay, demonstrating the sustained inhbibition of the protease by the RgpB propeptide. The negative control was 1 mM TLCK (FIG. 14).

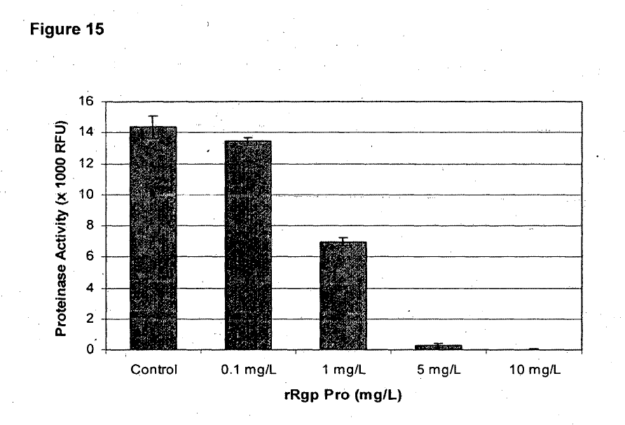

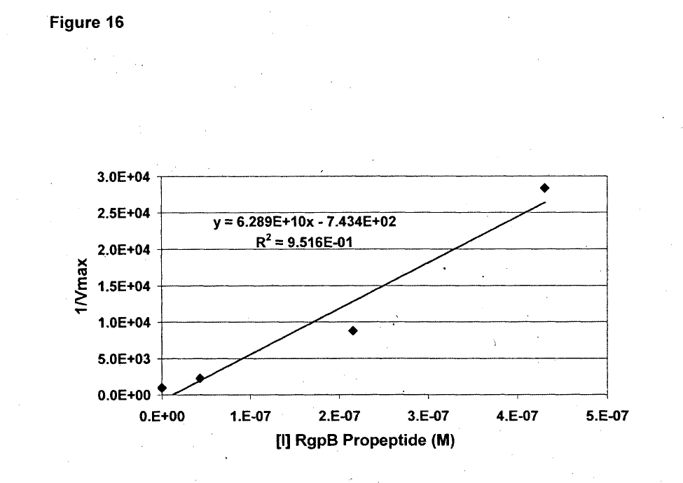

A dose response of the RgpB propeptide was demonstrated within a 2 h, incubation period where 1 mg/L inhibited ˜50% of RgpB activity whilst 5 mg/L totally abolished activity (FIG. 15). Inhibition kinetics of the RgpB propeptide were determined using the chromogenic substrate BapNA (FIG. 16). The Ki′ for non-competitive inhibition was calculated to be 11.8 nM.

### Propeptide Selectivity and Specificity

Both rRgpB and rKgp propeptides demonstrated selectivity for their cognate protease with no inhibition observed when rKgp propeptides were incubated with RgpB and vice versa (Table 1). The specificity of the propeptides was further examined using two examples of cysteine proteases. The clan CA protease papain, with a propeptide of 115 residues, was not significantly inhibited by rKgp and rRgpB propeptides (Table 1). The Clan CD protease caspase 3 that has structural homology with the RgpB and Kgp catalytic domains also was not inhibited by either rKgp or rRgpB propeptides. The non-competitive inhibition mode demonstrated by both propeptides, coupled with the selectivity for the cognate proteases is suggestive of exosite binding by the propeptides.

TABLE 1

| | | | | [Inhibitor] | | % Proteolytic |
|---|---|---|---|---|---|---|
| Protease | [Protease] | | Inhibitor | (mg/L) | Substrate | Activity |
| RgpB | 0.0085 | mg/ml | Kgp-PP | 50 | BapNa | 105 |
| Kgp | 0.0075 | mg/ml | RgpB-PP | 50 | GPKNa | 118 |
| Caspase 3 | 60 | units | Kgp-PP | 100 | Ac-DEVD-pNa | 121.1 ± 6.8 |
| | | | | | (200 uM) | |
| Caspase 3 | 60 | units | RgpB-PP | 100 | Ac-DEVD-pNa | 128.7 ± 6.2 |
| | | | | | (200 uM) | |
| Papain | 2.75 | mg/ml | Kgp-PP | 40 | BapNa | 88 |
| Papain | 2.75 | mg/ml | RgpB-PP | 40 | BapNa | 68 |

| | | | | | |
|---|---|---|---|---|---|
| Whole cell | 3.2 × 10⁷ | Kgp-PP | 40 | GPKNa | 65 |
| W50 | cells | | 80 | | 40 |
| Whole cell | 3.2 × 10⁷ | RgpB-PP | 40 | BapNa | 68 |
| W50 | cells | | 80 | | 59 |

### Planktonic Growth Inhibition

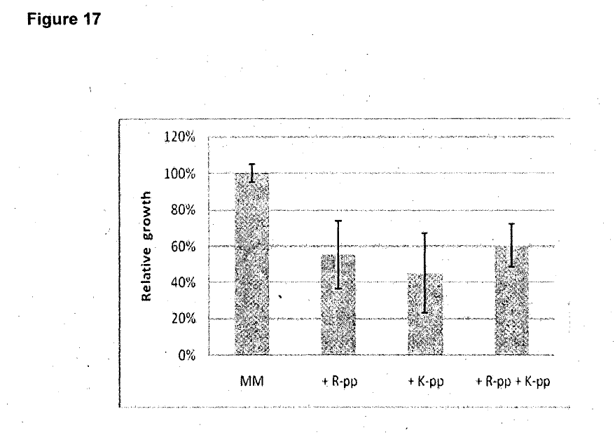

P. gingivalis W50 was grown in a protein-based minimal medium and reached a maximum cell density equivalent to an OD₆₂₀ₙₘ of 0.32 after 40 h of incubation. Both propeptides demonstrated a significant inhibitory effect on P. gingivalis W50 planktonic growth (FIG. 17). The P. gingivalis triple protease mutant lacking the RgpA, RgpB and Kgp gingipains, did not grow in this minimal medium thus confirming that gingipain proteolytic activitiy is essential for the breakdown of the proteins BSA and haemoglobin to short peptides for subsequent uptake by the bacterium.

### EXAMPLE 3

### Compositions and Formulations

To help illustrate compositions embodying aspects of the invention directed to treatment or prevention, the following sample formulations are provided.

The following is an example of a toothpaste formulation.

| Ingredient | % w/w |
|---|---|
| Dicalcium phosphate dihydrate | 50.0 |
| Glycerol | 20.0 |
| Sodium carboxymethyl cellulose | 1.0 |
| Sodium lauryl sulphate | 1.5 |
| Sodium lauroyl sarconisate | 0.5 |
| Flavour | 1.0 |
| Sodium saccharin | 0.1 |
| Chlorhexidine gluconate | 0.01 |
| Dextranase | 0.01 |
| Compound, peptide or peptidomimetic of the invention | 0.2 |
| Water | balance |

The following is an example of a further toothpaste formulation.

| Ingredient | % w/w |
|---|---|
| Dicalcium phosphate dihydrate | 50.0 |
| Sorbitol | 10.0 |
| Glycerol | 10.0 |
| Sodium carboxymethyl cellulose | 1.0 |
| Lauroyl diethanolamide | 1.0 |
| Sucrose monolaurate | 2.0 |
| Flavour | 1.0 |
| Sodium saccharin | 0.1 |
| Sodium monofluorophosphate | 0.3 |
| Chlorhexidine gluconate | 0.01 |
| Dextranase | 0.01 |
| Compound, peptide or peptidomimetic of the invention | 0.1 |
| Water | balance |

The following is an example of a further toothpaste formulation.

| Ingredient | % w/w |
|---|---|
| Sorbitol | 22.0 |
| Irish moss | 1.0 |
| Sodium Hydroxide (50%) | 1.0 |
| Gantrez | 19.0 |
| Water (deionised) | 2.69 |
| Sodium Monofluorophosphate | 0.76 |
| Sodium saccharine | 0.3 |
| Pyrophosphate | 2.0 |
| Hydrated alumina | 48.0 |
| Flavour oil | 0.95 |
| Compound, peptide or peptidomimetic of the invention | 0.3 |
| sodium lauryl sulphate | 2.00 |

The following is an example of a liquid toothpaste formulation.

| Ingredient | % w/w |
|---|---|
| Sodium polyacrylate | 50.0 |
| Sorbitol | 10.0 |
| Glycerol | 20.0 |
| Flavour | 1.0 |
| Sodium saccharin | 0.1 |
| Sodium monofluorophosphate | 0.3 |
| Chlorhexidine gluconate | 0.01 |
| Ethanol | 3.0 |
| Compound, peptide or peptidomimetic of the invention | 0.2 |
| Linolic acid | 0.05 |
| Water | balance |

The following is an example of a mouthwash formulation.

| Ingredient | % w/w |
|---|---|
| Ethanol | 20.0 |
| Flavour | 1.0 |
| Sodium saccharin | 0.1 |
| Sodium monofluorophosphate | 0.3 |
| Chlorhexidine gluconate | 0.01 |
| Lauroyl diethanolamide | 0.3 |
| Compound, peptide or peptidomimetic of the invention | 0.2 |
| Water | balance |

The following is an example of a further mouthwash formulation.

| Ingredient | % w/w |
|---|---|
| Gantrez ® S-97 | 2.5 |
| Glycerine | 10.0 |
| Flavour oil | 0.4 |
| Sodium monofluorophosphate | 0.05 |
| Chlorhexidine gluconate | 0.01 |
| Lauroyl diethanolamide | 0.2 |
| Compound, peptide or peptidomimetic of the invention | 0.3 |
| Water | balance |

The following is an example of a lozenge formulation.

| Ingredient | % w/w |
|---|---|
| Sugar | 75-80 |
| Corn syrup | 1-20 |
| Flavour oil | 1-2 |
| NaF | 0.01-0.05 |
| Compound, peptide or peptidomimetic of the invention | 0.3 |
| Mg stearate | 1-5 |
| Water | balance |

The following is an example of a gingival massage cream formulation.

| Ingredient | % w/w |
|---|---|
| White petrolatum | 8.0 |
| Propylene glycol | 4.0 |
| Stearyl alcohol | 8.0 |
| Polyethylene Glycol 4000 | 25.0 |
| Polyethylene Glycol 400 | 37.0 |
| Sucrose monostearate | 0.5 |
| Chlorhexidine gluconate | 0.1 |
| Compound, peptide or peptidomimetic of the invention | 0.3 |
| Water | balance |

The following is an example of a periodontal gel formulation.

| Ingredient | % w/w |
|---|---|
| Pluronic F127 (from BASF) | 20.0 |
| Stearyl alcohol | 8.0 |
| Compound, peptide or peptidomimetic of the invention | 3.0 |
| Colloidal silicon dioxide (such as Aerosil ® 200 ™) | 1.0 |
| Chlorhexidine gluconate | 0.1 |
| Water | balance |

The following is an example of a chewing gum formulation.

| Ingredient | % w/w |
|---|---|
| Gum base | 30.0 |
| Calcium carbonate | 2.0 |
| Crystalline sorbitol | 53.0 |
| Glycerine | 0.5 |
| Flavour oil | 0.1 |
| Compound, peptide or peptidomimetic of the invention | 0.3 |
| Water | balance |

It should be understood that while the invention has been described in detail herein, the examples are for illustrative purposes only. Other modifications of the embodiments of the present invention that are obvious to those skilled in the art of molecular biology, dental treatment, and related disciplines are intended to be within the scope of the invention.

### REFERENCES

Mizuguchi, K., C. M. Deane, et al. (1998). “HOMSTRAD: a database of protein structure alignments for homologous families.” Protein Sci 7(11): 2469-71.
Shi, J., T. L. Blundell, et al. (2001). “FUGUE: sequence-structure homology recognition using environment-specific substitution tables and structure-dependent gap penalties.” J Mol Biol 310(1): 243-57.
Stryer, L., J. M. Berg, et al. (2002). Biochemistry Fifth Edition. New York, W.H. Freeman and Company.
Kitano, S., Irimura, K., Sasaki, T., Abe, N., Baba, A., Miyake, Y., Katunuma, N. and Yamamoto, K. (2001). “Suppression of gingival inflammation induced by Porphyromonas gingivalis in rats by leupeptin.” Jpn J Pharmacol 85(1): 84-91.
Fletcher, H. M., Schenkein, H. A. and Macrina, F. L. (1994). “Cloning and characterization of a new protease gene (prtH) from Porphyromonas gingivalis.” Infect. Immun. 62(10): 4279-4286.
Pike, R., McGraw, W., Potempa, J. and Travis, J. (1994). “Lysine- and arginine-specific proteinases from Porphyromonas gingivalis. Isolation, characterization, and evidence for the existence of complexes with hemagglutinins.” J. Biol. Chem. 269(1): 406-411.
Zar, J. H. (1984). Biostatistical analysis. Englewood Cliffs, N.J., Prentice-Hall.
Fowler, J. and Cohen, L. (1997). Practical Statistics for field biology. Brisbane, Australia, John Wiley and Sons.
Gasteiger, E., Hoogland, C., Gattiker, A., Duvaud, S., Wilkins, M. R., Appel, R. D. and A., B. (2005). Protein Identification and Analysis Tools on the ExPASy Server. The Proteomics Protocols Handbook. J. M. Walker, Humana Press: 571-607.
Grimsley, G. R. and Pace, C. N. (2003). “Spectrophotometric Determination of Protein Concentration.” Current Protocols in Protein Science 33 (UNIT 3.1): 3.1.1-3.1.9.
Grenier, D., Imbeault, S., Plamondon, P., Grenier, G., Nakayama, K. and Mayrand, D. (2001). “Role of gingipains in growth of Porphyromonas gingivalis in the presence of human serum albumin.” Infect Immun 69(8): 5166-72.
Yoshioka, M., Grenier, D. and Mayrand, D. (2003). “Monitoring the uptake of protein-derived peptides by Porphyromonas gingivalis with fluorophore-labeled substrates.” Curr Microbiol 47(1): 1-4.

1. A peptide or peptidomimetic for inhibiting, reducing or preventing the activity of a bacterial enzyme, the compound, peptide or peptidomimetic consisting essentially of an amino acid sequence that is selected from the group consisting of SEQ ID NO: 1 to 28 and conservative substitutions therein.

2. A peptide or peptidomimetic for inhibiting, reducing or preventing the activity of a bacterial enzyme, the compound, peptide or peptidomimetic consisting of an amino acid sequence that is selected from the group consisting of SEQ ID NO: 1 to 28 and conservative substitutions therein.

3. A method of treating or preventing periodontal disease comprising administering to a subject an effective amount of a peptide or peptidomimetic according to claim 1.

4. Use of a peptide or peptidomimetic according to claim 1 in the manufacture of a medicament for the treatment or prevention of periodontal disease.

5. An assay for identifying an inhibitor of a cysteine protease comprising the steps of:
contacting a cysteine protease with a candidate compound in the presence of a peptide or peptidomimetic,
determining whether the candidate compound competes with the peptide or peptidomimetic;
wherein competition indicates that the candidate compound is an inhibitor of a cysteine protease,
wherein the peptide or peptidomimetic comprises an amino acid sequence of a gingipain propeptide or fragment thereof.

6. An assay according to claim 5, wherein the gingipain propeptide is selected from the group consisting of RgpA, RgpB and Kgp.

7. An assay according to claim 5 wherein the amino acid sequence of the propeptide is selected from the group consisting of SEQ ID NO: 1 to 28 and conservative substitutions therein.

8. An assay according to claim 5 wherein the propeptide or fragment thereof occurs naturally.

9. An assay according to claim 5 wherein the propeptide or fragment thereof is derived from P. gingivalis.

10. An assay according to claim 5 wherein the cysteine protease is a gingipain.

11. An assay according to claim 10, wherein the gingipain is selected from the group consisting of RgpA, RgpB and Kgp.

12. Use of a compound identified as an inihibtor of a cysteine protease by an assay according to claim 5 to treat or prevent periodontal disease

13. A compound, peptide or peptidomimetic comprising an amino acid sequence of a gingipain propeptide or fragment thereof for use in an assay according to claim 5.
